(19) United States
(12) Patent Application Publication
(10) Pub. No.: US 2010/0041103 A1
(43) Pub. Date: Feb. 18, 2010

(54) COMPOSITION AND METHOD FOR ENHANCING CELL GROWTH AND CELL FUSION

(75) Inventor: Eran GABBAI, Kfar=MaAs (IL)

Correspondence Address: FOLEY AND LARDNER LLP;SUITE 500, 3000 K STREET NW, WASHINGTON, DC, 20007, US

(73) Assignee: DO-COOP TECHNOLOGIES LTD.

(21) Appl. No.: 12/521,972

(22) PCT Filed: Jan. 3, 2008
(86) PCT No.: PCT/IL08/00025

§ 371 Date: Jul. 1, 2009

Related U.S. Application Data
(60) Provisional application No. 60/878,387, filed on Jan. 4, 2007.
(60) Provisional application No. 60/935,058, filed on Jul. 24, 2007.

(51) Int. Cl. C12N 5/02; C12N 15/02; C12P 21/00

(52) U.S. Cl. 435/69.6; 435/449; 435/325; 435/404

(57) ABSTRACT

A method of cell-fusion is provided, the method comprising fusing cells in a medium comprising a liquid composition having a liquid and nanostructures, each of the nanostructures comprising a core material of a nanometric size surrounded by an envelope of ordered fluid molecules, the core material and the envelope of ordered fluid molecules being in a steady physical state, thereby fusing cells. Compositions and articles of manufacture are also provided for generating monoclonal antibodies and culturing eukaryotic cells.

### FIELD AND BACKGROUND OF THE INVENTION

The present invention relates to novel compositions for enhancing cell growth and cell fusion.

The living body of a mammal possesses humoral immunity which is a defense system for specifically capturing and eliminating exogenous antigens (e.g. viruses, bacterial toxins, and chemical substances), autoantigens (e.g. autoreactive lymphocytes; cancer cells and excessive endogenous factors (e.g. cytokines, hormones, or growth factors) which are detrimental for maintaining homeostasis in the living body and can become pathogenic causing or adding to the deterioration of various diseases. In this humoral immunity, the antibodies play a major role.

An antibody has a Y-shaped basic structure comprising four polypeptide chains—two long polypeptide chains (immunoglobulin heavy chains; IgH chains) and two short polypeptide chains (immunoglobulin light chains; IgL chains). The Y-shaped structure is made when the two IgH chains bridged by disulfide bonds are connected to each of the IgL chains through another disulfide bond.

Due to this function of capturing and eliminating antigens harmful to the living body, antibodies have been used as drugs for a long period of time. Polyclonal antibodies were the earliest forms of antibody drugs, where antiserum comprising various types of antibodies against a specific antigen, were used. The method for obtaining this antiserum, however was limited to collecting from sera, and therefore, the supply was inevitably limited. Moreover, it was extremely difficult to isolate a single type of antibody molecule comprising specificity to an antigen, from this antiserum.

The successful preparation of a monoclonal antibody using hybridoma by Kohler and Milstein in 1975 (Nature, Vol. 256, p. 495-497, 1975) led to the solution of these problems and opened the doors for monoclonal antibodies to be used as drugs since it became possible to generate an antibody to a specific antigen on demand.

Typically, the production of human monoclonal antibodies requires the immortalization of human B-lymphocytes by fusion with a partner cell-line of a myeloid source. The results of these cell fusions are named “hybridomas” which possess the qualities of both parental cell-lines: the ability to grow continually, and the ability to produce pure antibody.

However, since the only human B-cells that are available for monoclonal antibody production are the ones that circulate in the peripheral blood, the source of cells for monoclonal antibody production is limited. Furthermore, although theoretically possible, it is hard to produce human monoclonal antibodies against antigens if the immune response that they caused was not recent or recurring. In addition, it has proven difficult to produce high levels of isolated monoclonal antibodies from a hybridoma cell culture as the quantities of secreted monoclonal antibodies are typically not high.

In order to bridge the theoretical and the practical outcomes of monoclonal antibody production, the efficiency of the fusion process needs to be very high, to overcome the rarity of the B-cells obtained from peripheral blood, thus making their chances of immortalization higher.

There is thus a widely recognized need for, and it would be highly advantageous to have methods of producing large amounts of monoclonal antibodies in a cost effective manner.

### SUMMARY OF THE INVENTION

According to an aspect of some embodiments of the present invention there is provided a method of cell-fusion, the method comprising fusing cells in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state, thereby fusing cells.

According to an aspect of some embodiments of the present invention there is provided a method of culturing eukaryotic cells, the method comprising incubating the cells in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state, thereby culturing eukaryotic cells.

According to an aspect of some embodiments of the present invention there is provided a cell culture medium comprising a eukaryotic cell culture medium and a liquid composition having a liquid and nanostructures, each of said nanostructures comprising a core material of a nanometric size surrounded by an envelope of ordered fluid molecules, said core material and said envelope of ordered fluid molecules being in a steady physical state.

According to an aspect of some embodiments of the present invention there is provided an article of manufacture comprising packaging material and a liquid composition identified for the culturing of eukaryotic cells being contained within said packaging material, said liquid composition having a liquid and nanostructures, each of said nanostructures comprising a core material of a nanometric size surrounded by an envelope of ordered fluid molecules, said core material and said envelope of ordered fluid molecules being in a steady physical state.

According to an aspect of some embodiments of the present invention there is provided an article of manufacture comprising packaging material and a liquid composition identified for generating monoclonal antibodies being contained within said packaging material, said liquid composition having a liquid and nanostructures, each of said nanostructures comprising a core material of a nanometric size surrounded by an envelope of ordered fluid molecules, said core material and said envelope of ordered fluid molecules being in a steady physical state.

According to an aspect of some embodiments of the present invention there is provided a method of generating a monoclonal antibody, the method comprising fusing an immortalizing cell with an antibody producing cell to obtain a hybridoma in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state.

According to an aspect of some embodiments of the present invention there is provided a method of dissolving or dispersing cephalosporin comprising contacting the cephalosporin with nanostructures and liquid under conditions which allow dispersion or dissolving of the substance, wherein said nanostructures comprise a core material of a nanometric size enveloped by ordered fluid molecules of said liquid, said core material and said envelope of ordered fluid molecules being in a steady physical state.

According to some embodiments of the invention, the cells are identical.

According to some embodiments of the invention, the cells are non-identical.

According to some embodiments of the invention, the cells comprise primary cells.

According to some embodiments of the invention, the cells comprise immortalized cells.

According to some embodiments of the invention, the non-identical cells comprise tumor cells and antibody producing cells.

According to some embodiments of the invention, the non-identical cells comprise stem cells and somatic cells.

According to some embodiments of the invention, the stem cells are embryonic stem cells.

According to some embodiments of the invention, the somatic cells are muscle cells or bone cells.

According to some embodiments of the invention, the antibody producing cells are B lymphocytes.

According to some embodiments of the invention, the B lymphocytes are human B lymphocytes.

According to some embodiments of the invention, the B lymphocytes are peripheral blood mononuclear cells.

According to some embodiments of the inventions the tumor cells are incubated in said liquid composition for a period of time which allows an increase in hybridoma generation prior to said fusing.

According to some embodiments of the invention, the period of time is no less than one day.

According to some embodiments of the invention, at least a portion of said fluid molecules are identical to molecule of said liquid.

According to some embodiments of the invention, the at least a portion of said fluid molecules are in a gaseous state.

According to some embodiments of the invention, a concentration of said nanostructures is lower than 10²⁰ nanostructures per liter.

According to some embodiments of the invention, the nanostructures are capable of forming clusters of said nanostructures.

According to some embodiments of the invention, the nanostructures are capable of maintaining long range interaction thereamongst.

According to some embodiments of the invention, the liquid composition comprises a buffering capacity greater than a buffering capacity of water.

According to some embodiments of the invention, the liquid composition is formulated from hydroxyapatite.

According to some embodiments of the invention, the liquid composition is capable of altering polarization of light.

According to some embodiments of the invention, the medium further comprises at least one agent selected from the group consisting of a growth factor, a serum and an antibiotic.

According to some embodiments of the invention, the eukaryotic cells are single cells.

According to some embodiments of the invention, the single cell is a hybridoma.

According to some embodiments of the invention, the culturing is effected in the absence of HCF.

According to some embodiments of the invention, the eukaryotic cells are mesenchymal stem cells.

According to some embodiments of the invention, the eukaryotic cell culture medium further comprises at least one agent selected from the group consisting of a growth factor, a serum and an antibiotic.

According to some embodiments of the invention, the liquid composition is capable of increasing a cell proliferation rate.

According to some embodiments of the invention, the method further comprises cloning said hybridoma.

According to some embodiments of the invention, the cloning is effected by incubating said hybridoma in a medium comprising said liquid composition.

According to some embodiments of the invention, the cloning is effected in the absence of HCF.

According to some embodiments of the invention, the method further comprises harvesting the monoclonal antibody following said cloning.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph illustrating the proliferation of bone marrow cells in MEM medium based on Neowater™ of RO (reverse osmosis) water.

FIG. 2 is a graph illustrating Sodium hydroxide titration of various water compositions as measured by absorbance at 557 nm.

FIGS. 3A-C are graphs of an experiment performed in triplicate illustrating Sodium hydroxide titration of water comprising nanostructures and RO water as measured by pH.

FIGS. 4A-C are graphs illustrating Sodium hydroxide titration of water comprising nanostructures and RO water as measured by pH, each graph summarizing 3 triplicate experiments.

FIGS. 5A-C are graphs of an experiment performed in triplicate illustrating Hydrochloric acid titration of water comprising nanostructures and RO water as measured by pH.

FIG. 6 is a graph illustrating Hydrochloric acid titration of water comprising nanostructures and RO water as measured by pH, the graph summarizing 3 triplicate experiments.

FIGS. 7A-C are graphs illustrating Hydrochloric acid (FIG. 10A) and Sodium hydroxide (FIGS. 10B-C) titration of water comprising nanostructures and RO water as measured by absorbance at 557 nm.

FIGS. 8A-B are photographs of cuvettes following Hydrochloric acid titration of RO (FIG. 8A) and water comprising nanostructures (FIG. 8B). Each cuvette illustrated addition of 1 μl of Hydrochloric acid.

FIGS. 9A-C are graphs illustrating Hydrochloric acid titration of RF water (FIG. 9A), RF2 water (FIG. 9B) and RO water (FIG. 9C). The arrows point to the second radiation.

FIG. 10 is a graph illustrating Hydrochloric acid titration of FR2 water as compared to RO water. The experiment was repeated three times. An average value for all three experiments was plotted for RO water.

FIGS. 11A-J are photographs of solutions comprising red powder and Neowater™ following three attempts at dispersion of the powder at various time intervals. FIGS. 11A-E illustrate right test tube C (50% EtOH+Neowater™) and left test tube B (dehydrated Neowater™) from Example 8 part C. FIGS. 11G-J illustrate solutions following overnight crushing of the red powder and titration of 100 μl Neowater™

FIGS. 12A-C are readouts of absorbance of 2 μl from 3 different solutions as measured in a nanodrop. FIG. 12A represents a solution of the red powder following overnight crushing+100 μl Neowater. FIG. 12B represents a solution of the red powder following addition of 100% dehydrated Neowater™ and FIG. 12C represents a solution of the red powder following addition of EtOH+Neowater™ (50%-50%).

FIG. 13 is a graph of spectrophotometer measurements of vial #1 (CD-Dau+Neowater™), vial #4 (CD-Dau+10% PEG in Neowater™) and vial #5 (CD-Dau+50% Acetone+50% Neowater™).

FIG. 14 is a graph of spectrophotometer measurements of the dissolved material in Neowater™ (blue line) and the dissolved material with a trace of the solvent acetone (pink line).

FIG. 15 is a graph of spectrophotometer measurements of the dissolved material in Neowater™ (blue line) and acetone (pink line). The pale blue and the yellow lines represent different percent of acetone evaporation and the purple line is the solution without acetone.

FIG. 16 is a graph of spectrophotometer measurements of CD-Dau at 200-800 nm. The blue line represents the dissolved material in RO while the pink line represents the dissolved material in Neowater™.

FIG. 17 is a graph of spectrophotometer measurements of t-boc at 200-800 nm. The blue line represents the dissolved material in RO while the pink line represents the dissolved material in Neowater™.

FIGS. 18A-D are graphs of spectrophotometer measurements at 200-800 nM. FIG. 18A is a graph of AG-14B in the presence and absence of ethanol immediately following ethanol evaporation. FIG. 18B is a graph of AG-14B in the presence and absence of ethanol 24 hours following ethanol evaporation. FIG. 18C is a graph of AG-14A in the presence and absence of ethanol immediately following ethanol evaporation. FIG. 18D is a graph of AG-14A in the presence and absence of ethanol 24 hours following ethanol evaporation.

FIG. 19 is a photograph of suspensions of AG-14A and AG14B 24 hours following evaporation of the ethanol.

FIGS. 20A-G are graphs of spectrophotometer measurements of the peptides dissolved in Neowater™. FIG. 20A is a graph of Peptide X dissolved in Neowater™. FIG. 20B is a graph of X-5FU dissolved in Neowater™. FIG. 20C is a graph of NLS-E dissolved in Neowater™. FIG. 20D is a graph of Palm-PFPSYK (CMFU) dissolved in Neowater™. FIG. 20E is a graph of PFPSYKLRPG-NH₂ dissolved in Neowater™. FIG. 20F is a graph of NLS-p2-LHRH dissolved in Neowater™, and FIG. 20G is a graph of F-LH-RH-palm kGFPSK dissolved in Neowater™.

FIGS. 21A-G are bar graphs illustrating the cytotoxic effects of the peptides dissolved in Neowater™ as measured by a crystal violet assay. FIG. 21A is a graph of the cytotoxic effect of Peptide X dissolved in Neowater™. FIG. 21B is a graph of the cytotoxic effect of X-5FU dissolved in Neowater™. FIG. 21C is a graph of the cytotoxic effect of NLS-E dissolved in Neowater™. FIG. 21D is a graph of the cytotoxic effect of Palm-PFPSYK (CMFU) dissolved in Neowater™. FIG. 21E is a graph of the cytotoxic effect of PFPSYKLRPG-NH₂ dissolved in Neowater™. FIG. 21F is a graph of the cytotoxic effect of NLS-p2-LHRH dissolved in Neowater™, and FIG. 21G is a graph of the cytotoxic effect of F-LH-RH-palm kGFPSK dissolved in Neowater™.

FIG. 22 is a graph of retinol absorbance in ethanol and Neowater™.

FIG. 23 is a graph of retinol absorbance in ethanol and Neowater™ following filtration.

FIGS. 24A-B are photographs of test tubes, the left containing Neowater™ and substance “X” and the right containing DMSO and substance “X”. FIG. 24A illustrates test tubes that were left to stand for 24 hours and FIG. 24B illustrates test tubes that were left to stand for 48 hours.

FIGS. 25A-C are photographs of test tubes comprising substance “X” with solvents 1 and 2 (FIG. 28A), substance “X” with solvents 3 and 4 (FIG. 25B) and substance “X” with solvents 5 and 6 (FIG. 25C) immediately following the heating and shaking procedure.

FIGS. 26A-C are photographs of test tubes comprising substance “X” with solvents 1 and 2 (FIG. 26A), substance “X” with solvents 3 and 4 (FIG. 26B) and substance “X” with solvents 5 and 6 (FIG. 26C) 60 minutes following the heating and shaking procedure.

FIGS. 27A-C are photographs of test tubes comprising substance “X” with solvents 1 and 2 (FIG. 27A), substance “X” with solvents 3 and 4 (FIG. 27B) and substance “X” with solvents 5 and 6 (FIG. 27C) 120 minutes following the heating and shaking procedure.

FIGS. 28A-C are photographs of test tubes comprising substance “X” with solvents 1 and 2 (FIG. 28A), substance “X” with solvents 3 and 4 (FIG. 28B) and substance “X” with solvents 5 and 6 (FIG. 28C) 24 hours following the heating and shaking procedure.

FIGS. 29A-D are photographs of glass bottles comprising substance “X” in a solvent comprising Neowater™ and a reduced concentration of DMSO, immediately following shaking (FIG. 29A), 30 minutes following shaking (FIG. 29B), 60 minutes following shaking (FIG. 29C) and 120 minutes following shaking (FIG. 29D).

FIG. 30 is a graph illustrating the absorption characteristics of material “X” in RO/Neowater™ 6 hours following vortex, as measured by a spectrophotometer.

FIGS. 31A-B are graphs illustrating the absorption characteristics of SPL2101 in ethanol (FIG. 31A) and SPL5217 in acetone (FIG. 31B), as measured by a spectrophotometer.

FIGS. 32A-B are graphs illustrating the absorption characteristics of SPL2101 in Neowater™ (FIG. 32A) and SPL5217 in Neowater™ (FIG. 32B), as measured by a spectrophotometer.

FIGS. 33A-B are graphs illustrating the absorption characteristics of taxol in Neowater™ (FIG. 33A) and DMSO (FIG. 33B), as measured by a spectrophotometer.

FIG. 34 is a bar graph illustrating the cytotoxic effect of taxol in different solvents on 293T cells. Control RO=medium made up with RO water; Control Neo=medium made up with Neowater™; Control DMSO RO=medium made up with RO water+10 μl DMSO; Control Neo RO=medium made up with RO water+10 μl Neowater™; Taxol DMSO RO=medium made up with RO water+taxol dissolved in DMSO; Taxol DMSO Neo=medium made up with Neowater™+taxol dissolved in DMSO; Taxol NW RO=medium made up with RO water+taxol dissolved in Neowater™; Taxol NW Neo=medium made up with Neowater™+taxol dissolved in Neowater™.

FIGS. 35A-B are photographs of a DNA gel stained with ethidium bromide illustrating the PCR products obtained in the presence and absence of the liquid composition comprising nanostructures following heating according to the protocol described in Example 16 using two different Taq polymerases.

FIG. 36 is a photograph of a DNA gel stained with ethidium bromide illustrating the PCR products obtained in the presence and absence of the liquid composition comprising nanostructures following heating according to the protocol described in Example 17 using two different Taq polymerases.

FIG. 37A is a graph illustrating the spectrophotometric readouts of 0.5 mM taxol in Neowater™ and in DMSO.

FIGS. 37B-C are HPLC readouts of taxol in Neowater™ and in DMSO. FIG. 37B illustrates the HPLC readout of a freshly prepared standard (DMSO) formulation of taxol. FIG. 37C illustrates the HPLC readout of taxol dispersed in Neowater™ after 6 months of storage at −20° C.

FIG. 38 is a bar graph illustrating PC3 cell viability of various taxol concentrations in DMSO or Neowater™ formulations. Each point represents the mean±standard deviation from eight replicates.

FIG. 39 is a bar graph illustrating fusion efficiency enhancement by Neowater™. The fusions were performed according to a standard protocol, where the culture media and PEG were reconstituted from powder forms with either Neowater™ (NPD) or control water (DI). For each fusion, PBMC from a single batch were divided into two equal fractures and used to prepare two parallel experiments, in Neowater™ or control water based reagents. The figure presents percents of hybridoma-positive wells in each fusion experiment. The percent was calculated as the number of hybridoma-positive wells from a 96-well plate where the cells were seeded and grown after the fusion process. The difference between all the Neowater™- and control water-fusion results was found to be statistically significant by Chi-square analysis (p<<0.001). The percent of enhancement was calculated by the formula [(number of hybridomas in Neowater ™-fusion/number of hybridomas in control water-fusion)×100%-100%]

FIG. 40 is a bar graph illustrating the cloning efficiency of a semi-stable clone in Neowater™(NPD) and control water (DI). From an antibody-producing semi-stable clone, 200 cells were counted and seeded in a volume of 10 mL over a 96-well plate (on average 1-2 cells/100 μL/well). The figure presents a mean percent of hybridoma-positive wells per cloning experiment. The error bars denote the standard error of the mean.

FIGS. 41A-B are bar graphs illustrating the ability of Neowater™ to enhance antibody secretion from a stable hybridoma clone in 10% FCS. Two parallel cultures were prepared in replicates from a stable hybridoma clone. One was grown in Neowater™ (NPD) and the other in control water (DI) medium and both were kept in standard culture conditions. After a week of growth the supernatants were collected, and the antibody concentrations were measured by a standard sandwich ELISA. Each column represents the mean antibody concentration that was measured in Neowater™ (NPD) and control water (DI) cultures. The error bars denote the standard error of the means. FIG. 41A illustrates the total antibody concentration measured in the culture supernatants; FIG. 41B illustrates the antibody concentration normalized per cell.

FIGS. 42A-B are graphs illustrating IGM production by a stable hybridoma clone in 3% FCS. Two cultures derived from the same culture of a stable hybridoma clone were grown, one in Neowater™ (NPD) and the other in control water (DI) based medium supplemented with 3% FCS. Before seeding, the cells were washed in serum-free media to verify the removal of any residual serum. During a period of two weeks the supernatants were collected as indicated and the cells were counted on the same day. The cultures were fed on the 4^{th} and 10^{th} day and medium was placed in the cultures on day 6. Although the cells in DI culture proliferated normally under these conditions, they failed to produce measurable quantities of antibody

FIGS. 43A-C are bar graphs illustrating CHO cell growth in reduced serum medium. FIG. 43A: Cells were seeded at an initial density of 1.5×10⁶ per 10-cm Petri dish in Neowater™ (NPD) and control water (DI) based medium in triplicates. After overnight growth they were detached by trypsinization and counted. The results are given as the number of viable cells. Each column represents a mean number of cells in each treatment. The error bars denote the standard error of the means. The difference between the treatments is 30%. The graph provides a representative result of an experiment, which was conducted with replicates and repeated three times.

FIGS. 43B, C: Cells were seeded in 96-well plates in multiple replicates (18 wells per treatment) in Neowater™ (NPD) or control water (DI) medium supplemented with 5% or 1% FCS. The results were quantified and analyzed by means of crystal violet dye retention assay. Each column represents the mean cell density following a given treatment in O.D. units. The error bars denote the standard error of the mean. *Significant difference between NPD and DI grown cells p=0.0006, total difference 7%. **Significant difference between NPD and DI grown cells p=0.0001, total difference 14%.

FIGS. 44A-B are bar graphs illustrating the effect of Neowater™ on primary human fibroblast proliferation. FIG. 44A. Primary human fibroblasts were seeded in replicate in a 96-well plate at two initial cell densities: five and ten thousand cells per well. After an overnight growth the cells were fixed and assayed by means of crystal violet dye retention method. The results are presented in O.D. values. Each column represents a mean O.D. of a given growth condition; the error bars denote the standard error of the mean. *Significant difference between DI (Control water) and NPD (Neowater™)for cell density of 5000 cells/well (p<<0.0001). **Significant difference between DI and NPD for cell density of 10000 cells/well (p<<0.0001). FIG. 44B. In a 24-well plate primary human fibroblasts were seeded in triplicate in NPD and DI based media. Next sets of triplicates (both in NPD and DI) were analyzed, by detaching and counting the viable cells, every 24 hours. The results are given in number of viable cells per well, the error bars denote the standard error of the mean.

FIG. 45 is a bar graph illustrating the effect of Neowater™ on mesencymal stem cell proliferation as measured by counting cell number.

FIG. 46 is a bar graph illustrating the effect of Neowater™ on mesencymal stem cell proliferation as measured by crystal violet stain

FIG. 47 is a spectrophotometer readout of cephalosporin dissolved in 100% acetone.

FIG. 48 is a spectrophotometer readout of Cephalosporin dissolved in Neowater™ prior to and following filtration.

FIGS. 49A-B are DH5α growth curves in LB with different Cephalosporin concentrations. Bacteria were grown at 37° C. and 220 rpm on two separate occasions.

FIGS. 50 A-B are bar graphs illustrating DH5α viability with two different Cephalosporin concentrations in reference to the control growth (no Cephalosporin added) 7 h post inoculation on two separate occasions (the control group contains 100 μl of Neowater™).

FIG. 51 is a graph illustrating the optical activity of Neowater™ relative to DDW spectrum. The red and blue curves are measurements of different Neowater™ batches, measured at different dates.

### DESCRIPTION OF THE PREFERRED EMBODIMENTS

The present invention is of novel compositions which can enhance both cell growth and cell fusion.

Specifically, the present invention can be used to enhance monoclonal antibody production.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The production of human monoclonal antibodies requires the immortalization of human B-lymphocytes by fusion with a partner cell-line of a myeloid source. However, since the only human B-cells that are available for monoclonal antibody production are the ones that circulate in the peripheral blood, the source of cells for monoclonal antibody production is limited.

In addition, it has proven difficult to produce high levels of isolated monoclonal antibodies from a hybridoma cell culture as the quantities of secreted monoclonal antibodies are typically not high.

In order to bridge the theoretical and the practical outcomes of monoclonal antibody production, the efficiency of the fusion process needs to be very high, to overcome the rarity of the B-cells obtained from peripheral blood, thus making their chances of immortalization higher. In addition methods need to be sought to enhance both the stability of hybridomas and secretion of monoclonal antibodies therefrom.

Whilst reducing the present invention to practice, the present inventors have uncovered that compositions comprising nanostructures (such as described in U.S. Pat. Appl. No. 60/545,955 and Ser. No. 10/865,955, and International Patent Application, Publication No. WO2005/079153) promote both cell fusion and cell stability.

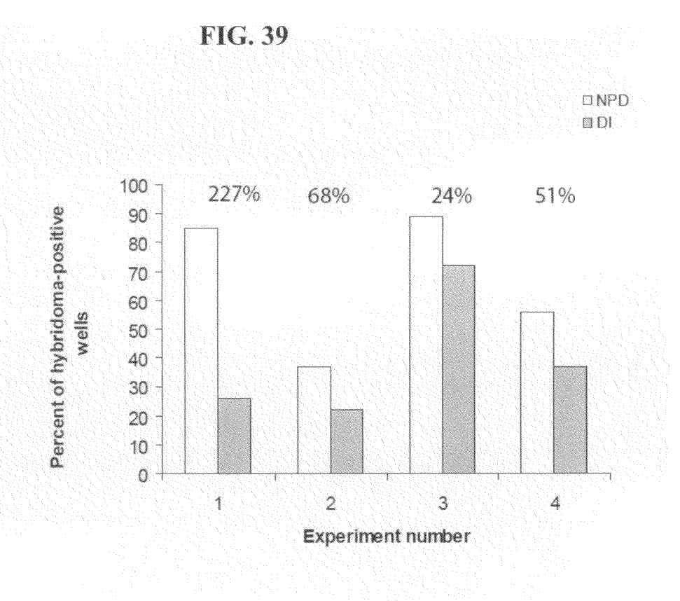

As illustrated hereinbelow and in the Examples section which follows the present inventors have demonstrated that nanostructures and liquid promote fusion of human peripheral blood mononuclear cells (PBMC) and fusion partner (MFP-2) cells and also promotes the stability of the hybridomas produced therefrom (see Tables 1 and 3 of Example 1 hereinbelow and FIG. 39 and Table 6 of Example 19). In addition the present inventors have shown that nanostructures and liquid increase antibody secretion from the hybridomas. Thus the liquid and nanostructures of the present invention may aid in the isolation and production of monoclonal antibodies.

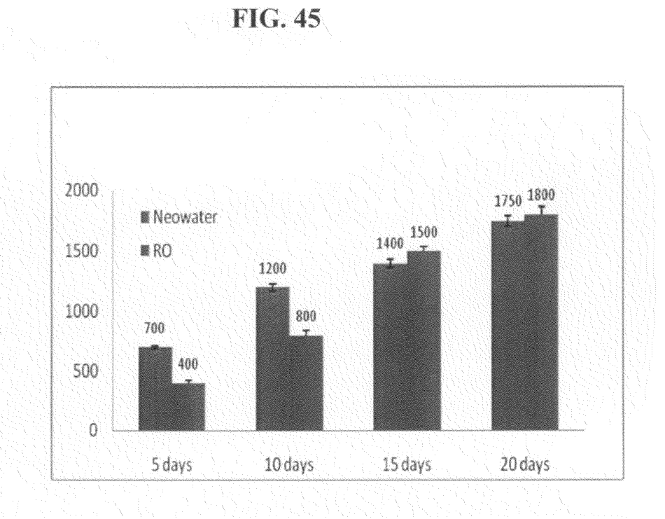

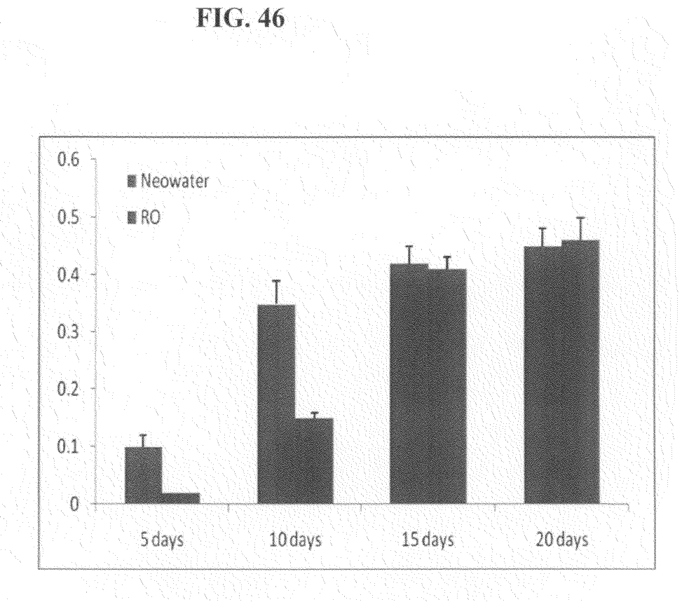

The present invention exploits this finding to provide novel compositions that promote not only monoclonal antibody production, but also enhance fusion between other eukaryotic cells as well as to enhance growth of cells in general and mesenchmal stem cells in particular (FIGS. 45-46).

Thus, according to one aspect of the present invention there is provided a method of cell-fusion, the method comprising fusing cells in a medium comprising a liquid composition having a liquid and nanostructures, each of the nanostructures comprising a core material of a nanometric size surrounded by an envelope of ordered fluid molecules, the core material and the envelope of ordered fluid molecules being in a steady physical state, thereby affecting cell-fusion.

As used herein the phrase “cell-fusion” refers to the merging, (either ex vivo or in vivo) of two or more viable cells.

Cell-fusion may be accomplished by any method of combining cells under fuseogenic conditions. For example cells may be fused in the presence of a fusion stimulus such as polyethylene glycol (PEG) or Sendai virus (See, for example, Harlow & Lane (1988) in Antibodies, Cold Spring Harbor Press, New York). Alternatively, cells may be fused under appropriate electrical conditions.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, in which case the nanostructure is referred to as a “continuous nanostructure”, or between several hundreds of picometers to several hundreds of nanometers, in which the nanostructure is referred to as a “discontinuous nanostructure”. Thus, the nanostructure of the present embodiments can comprise a nanoparticle, an arrangement of nanoparticles, or any arrangement of one or more nanoparticles and one or more molecules.

The liquid of the above-described composition is preferably an aquatic liquid e.g., water.

According to one preferred embodiment of this aspect of the present invention the nanostructures of the liquid composition comprise a core material of a nanometer size enveloped by ordered fluid molecules, which are in a steady physical state with the core material and with each other. Such a liquid composition is described in U.S. Pat. Appl. No. 60/545,955 and Ser. No. 10/865,955 and International Pat. Appl. Publication No. WO2005/079153 to the present inventor, the contents of which are incorporated herein by reference.

Examples of such core materials include, without being limited to, a ferroelectric material, a ferromagnetic material and a piezoelectric material. A ferroelectric material is a material that maintains, over some temperature range, a permanent electric polarization that can be reversed or reoriented by the application of an electric field. A ferromagnetic material is a material that maintains permanent magnetization, which is reversible by applying a magnetic field. Preferably, the nanostructures retains the ferroelectric or ferromagnetic properties of the core material, thereby incorporating a particular feature in which macro scale physical properties are brought into a nanoscale environment.

The core material may also have a crystalline structure.

As used herein, the phrase “ordered fluid molecules” refers to an organized arrangement of fluid molecules which are interrelated, e.g., having correlations thereamongst. For example, instantaneous displacement of one fluid molecule can be correlated with instantaneous displacement of one or more other fluid molecules enveloping the core material.

As used herein, the phrase “steady physical state” is referred to a situation in which objects or molecules are bound by any potential having at least a local minimum. Representative examples, for such a potential include, without limitation, Van der Waals potential, Yukawa potential, Lenard-Jones potential and the like. Other forms of potentials are also contemplated.

Preferably, the ordered fluid molecules of the envelope are identical to the liquid molecules of the liquid composition. The fluid molecules of the envelope may comprise an additional fluid which is not identical to the liquid molecules of the liquid composition and as such the envelope may comprise a heterogeneous fluid composition.

Due to the formation of the envelope of ordered fluid molecules, the nanostructures of the present embodiment preferably have a specific gravity that is lower than or equal to the specific gravity of the liquid.

The fluid molecules may be either in a liquid state or in a gaseous state or a mixture of the two.

A preferred concentration of the nanostrucutures is below 10²⁰ nanostructures per liter and more preferably below 10¹⁵ nanostructures per liter. Preferably a nanostructure in the liquid is capable of clustering with at least one additional nanostructure due to attractive electrostatic forces between them. Preferably, even when the distance between the nanostructures prevents cluster formation (about 0.5-10 ?m), the nanostructures are capable of maintaining long-range interactions.

Without being bound to theory, it is believed that the long-range interactions between the nanostructures lends to the unique characteristics of the liquid composition. One such characteristic is that the liquid composition of the present invention is able to enhance the fusion process between two cell types, as demonstrated in the Example section that follows. Furthermore, the liquid composition has been shown to enhance the stability of cells as demonstrated in Example 2 of the Examples section that follows. In addition, the liquid composition was shown to enhance antibody secretion from the hybridomas (Example 19).

Production of the nanostructures according to this aspect of the present invention may be carried out using a “top-down” process. The process comprises the following method steps, in which a powder (e.g., a mineral, a ceramic powder, a glass powder, a metal powder, or a synthetic polymer) is heated, to a sufficiently high temperature, preferably more than about 700 ?C. Examples of solid powders which are contemplated include, but are not limited to, BaTiO₃, WO₃ and Ba₂F₉O₁₂. Surprisingly, the present inventors have shown that hydroxyapetite (HA) may also be heated to produce the liquid composition of the present invention. Hydroxyapatite is specifically preferred as it is characterized by intoxicity and is generally FDA approved for human therapy.

It will be appreciated that many hydroxyapatite powders are available from a variety of manufacturers such as Sigma Aldrich and Clarion Pharmaceuticals (e.g. Catalogue No. 1306-06-5).

As shown in Table 4, liquid compositions based on HA, all comprised enhanced buffering capacities as compared to water.

The heated powder is then immersed in a cold liquid, (water), below its density anomaly temperature, e.g., 3 ?C or 2 ?C. Simultaneously, the cold liquid and the powder are irradiated by electromagnetic RF radiation, preferably above 500 MHz, 750 MHz or more, which may be either continuous wave RF radiation or modulated RF radiation.

It has been demonstrated by the present inventor that during the production process described above, some of the large agglomerates of the source powder disintegrate and some of the individual particles of the source powder alter their shape and become spherical nanostructures. It is postulated [Katsir et al., “The Effect of rf-Irradiation on Electrochemical Deposition and Its Stabilization by Nanoparticle Doping”, Journal of The Electrochemical Society, 154(4) D249-D259, 2007] that during the production process, nanobubbles are generated by the radiofrequency treatment and cavitation is generated due to the injection of hot particles into water below the anomaly temperature. Since the water is kept below the anomaly temperature, the hot particles cause local heating that in turn leads to a local reduction of the specific volume of the heated location that in turn causes under pressure in other locations. It is postulated that during the process and a time interval of a few hours or less following the process, the water goes through a self-organization process that includes an exchange of gases with the external atmosphere and selective absorption of the surrounding electromagnetic radiation. It is further postulated that the self-organization process leads to the formation of the stable structured distribution composed of the nanobubbles and the nanostructures.

As demonstrated in the Examples section that follows, the liquid composition of the present embodiments is characterized by a non-vanishing circular dichroism signal. Circular dichroism is an optical phenomenon that results when a substance interacts with plane polarized light at a specific wavelength. Circular dichroism occurs when the interaction characteristics of one polarized-light component with the substance differ from the interaction characteristics of another polarized-light component with the substance. For example, an absorption band can be either negative or positive depending on the differential absorption of the right and left circularly polarized components for the substance.

It is recognized that non-vanishing circular dichroism signal of the liquid composition indicates that the liquid composition is an optically active medium. Thus, the liquid composition of the present embodiments can alter the polarization of light while interacting therewith. The present inventor postulates that the optical activity of the liquid composition of the present embodiments is a result of the long-range order which is manifested by the aforementioned formation of stable structured distribution of nanobubbles and nanostructures.

As mentioned hereinabove the liquid composition of the present invention was shown to aid in the process of cell-fusion. Examples of cells include primary cells and immortalized cells, identical cells and non-identical cells, human cells and non-human cells.

The phrase “immortalized cells” refers to cells or cell lines that can be passaged in cell culture for several generations or indefinitely. An example of an immortalized cell is a tumor cell.

Thus, for example, the liquid composition of the present invention may be used to assist in the ex vivo fusion between tumor cells and antibody producing cells (e.g. B lymphocytes) to produce a hybridoma The term “hybridoma” as used herein refers to a cell that is created by fusing two cells, a secreting cell from the immune system, such as a B-cell, and an immortal cell, such as a myeloma, within a single membrane. The resulting hybrid cell can be cloned, producing identical daughter cells. Each of these daughter clones can secrete cellular products of the immune cell over several generations.

According to a preferred embodiment of this aspect of the present invention, the B lymphocytes are human B lymphocytes. According to another preferred embodiment of this aspect of the present invention, the B lymphocytes are those which circulate in the peripheral blood e.g. PBMCs.

Examples of tumor cells which may be used to produce hybridomas according to this aspect of the present invention include mouse myeloma cells and cell lines, rat myeloma cell lines and human myeloma cell lines.

Preferably, the myeloma cell lines comprise a marker so a selection procedure may be established. For example the myeloma cell lines may be HGPRT negative (Hypoxanthine-guanine phosphoribosyl transferase) negative. Specific examples thereof include: X63-Ag8(X63), NS1-Ag4/1(NS-1), P3X63-Ag8.UI(P3UI), X63-Ag8.653(X63.653), SP2/0-Ag14(SP2/0), MPC11-45.6TG1.7(45.6TG), FO, S149/5XXO.BU.1, which are derived from mice; 210.RSY3.Ag.1.2.3(Y3) derived from rats; and U266AR(SKO-007), GM1500 GTG-A12(GM1500), UC729-6, LICR-LOW-HMy2(HMy2), 8226AR/NIP4-1(NP41) and MFP-2, which are derived from humans.

According to this aspect of the present invention, the tumor cells and/or B lymphocytes are incubated in a medium (e.g. a culture medium) comprising the liquid composition of the present invention.

As used herein the phrase “culture medium” refers to a medium having a composition which allows eukaryotic cells to remain viable for at least 12 hours and preferably to replicate.

Incubation in the liquid composition of the present invention may be effected prior to during and/or following the fusion procedure in order to increase the number of hybridomas. Incubation in the liquid composition of the present invention prior to the fusion process may be effected for any length of time so as to enhance hybridoma generation. Preferably, incubation is for more than one day. As illustrated in Example 1 herein below, MFP-2 cells (myeloma cells) were grown in a cell medium comprising the liquid composition of the present invention for approximately 20 days prior to fusion. The fusion procedure itself was also effected in medium comprising the liquid composition of the present invention.

According to any of the aspects of the present invention, the liquid portion of a culturing medium may be wholly or partly exchanged for the liquid composition of the present composition as further described hereinbelow.

The culture medium, according to any of the aspects of the present invention is typically selected on an empirical basis since each cell responds to a different culture medium in a particular way. Examples of culture medium are further described hereinbelow.

The liquid composition of the present invention may be used to aid in the ex-vivo fusion between other cells such as tumor cells and dendritic cells. It has been shown that such fused cells may be effective as anti-cancer vaccines [Zhang K et al., World J Gastroenterol. 2006 Jun. 7; 12(21):3438-41].

The liquid composition of the present invention may be used to aid in the in vivo fusion between somatic cells and stem cells. Because of their powerful generative and regenerative abilities, stem cells may be used to repair damage in the bone marrow and to different organs such as the liver, brain and heart. It has been shown that some of the stem cells' repair properties come from their ability to fuse with cells that are naturally resident in the organs they are repairing [Wang et al., 2003, Nature 422, 897-901]. Accordingly, the liquid composition of the present invention may be used to enhance fusion between stem cells and somatic cells such as bone cells and muscle cells. Thus, stem cells may be treated with the liquid composition of the present invention so that they fuse quicker and more efficiently to a target site, thereby directing the stem-cell repair process.

The liquid composition of the present invention may also be used for in vivo transferring nucleic acids by way of cell-fusion. See e.g., Hoppe U C, Circ Res. 1999 Apr. 30; 84(8):964-72

Another ex vivo fusion process which may be aided by the composition of the present invention is the fusion between embryonic stem cells and human cells. Such fusions were shown to generate hybrids which behaved in a similar manner to embryonic stem cells, thus generating genetically matched stem cells for transplants. Specifically, human embryonic stem (hES) cells were fused with human fibroblasts, resulting in hybrid cells that maintain a stable tetraploid DNA content and have morphology, growth rate, and antigen expression patterns characteristic of hES cells [Cowan et al., Science, 2005 Aug. 26; 309(5739):1369-73].

Yet another ex vivo fusion process which may be facilitated by the composition of the present invention is somatic cell nuclear transfer. This is the process by which a somatic cell is fused with an enucleated oocyte. The nucleus of the somatic cell provides the genetic information, while the oocyte provides the nutrients and other energy-producing materials that are necessary for development of an embryo. This procedure is used for cloning and generation of embryonic stem cells.

Whilst further reducing the invention to practice, the present inventors have shown that the liquid composition of the present invention enhances the whole process of monoclonal antibody production including the fusion process, the cloning of hybridomas generated thereby and the secretion of antibodies therefrom. The present inventors have shown that cloned hybridomas generated in the presence of the liquid composition of the present invention are more stable than cloned hybridomas generated in the absence of the liquid composition of the present invention.

Thus, according to another aspect of the present invention, there is provided a method of generating a monoclonal antibody, the method comprising fusing an immortalizing cell with an antibody producing cell to obtain a hybridoma in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state.

As used herein, the phrase “monoclonal antibody” refers to an immune molecule that comprises a single binding affinity for any antigen with which it immunoreacts.

According to this aspect of the present invention, monoclonal antibodies are generated by fusing an immortalizing cell with an antibody producing cell to produce hybridomas in the liquid composition of the present invention as described hereinabove. The generated hybridomas may then be cloned. According to a preferred embodiment of this aspect of the present invention, the cloning is effected by incubating single hybridomas in a medium comprising the liquid composition of the present invention.

Since cloned hybridomas generated in the presence of the liquid composition of the present invention are more stable than those generated in the absence thereof, the cloning procedure typically does not require the addition of a stabilizing factor such as HCF.

Following generation of hybridomas and optional cloning thereof, monoclonal antibodies may be screened and harvested. Many methods of screening are known in the art including functional and structural assays. An exemplary method for screening hybridomas is described in Example 2 hereinbelow using a sandwich ELISA assay.

Techniques for harvesting of monoclonal antibodies are also well known in the art and typically comprise standard protein purification methods.

According to yet a further aspect of the present invention, there is provided an article-of-manufacture, which comprises the composition of the present invention as described hereinabove, being packaged in a packaging material and identified in print, in or on the packaging material for use in generation of monoclonal antibodies, as described herein.

Since the composition of the present invention has been shown to enhance stabilization of eukaryotic cellular matter such as the hybridomas described hereinabove, the present inventors have realized that the composition of the present invention may be exploited to enhance stabilization of other eukaryotic cellular matter.

Thus, according to yet another aspect of the present invention there is provided a method of culturing eukaryotic cells. The method comprises incubating the cells in a medium comprising a liquid composition having a liquid and nanostructures, each of the nanostructures comprising a core material of a nanometric size surrounded by an envelope of ordered fluid molecules, the core material and the envelope of ordered fluid molecules being in a steady physical state.

Without being bound to theory, the present inventors believe that the liquid composition of the present invention is particularly appropriate for use in a culture medium for a number of reasons.

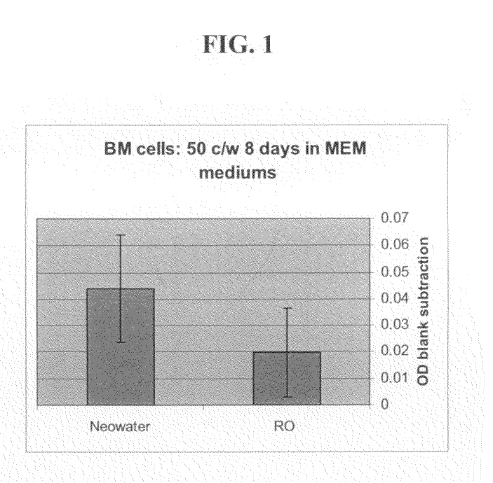

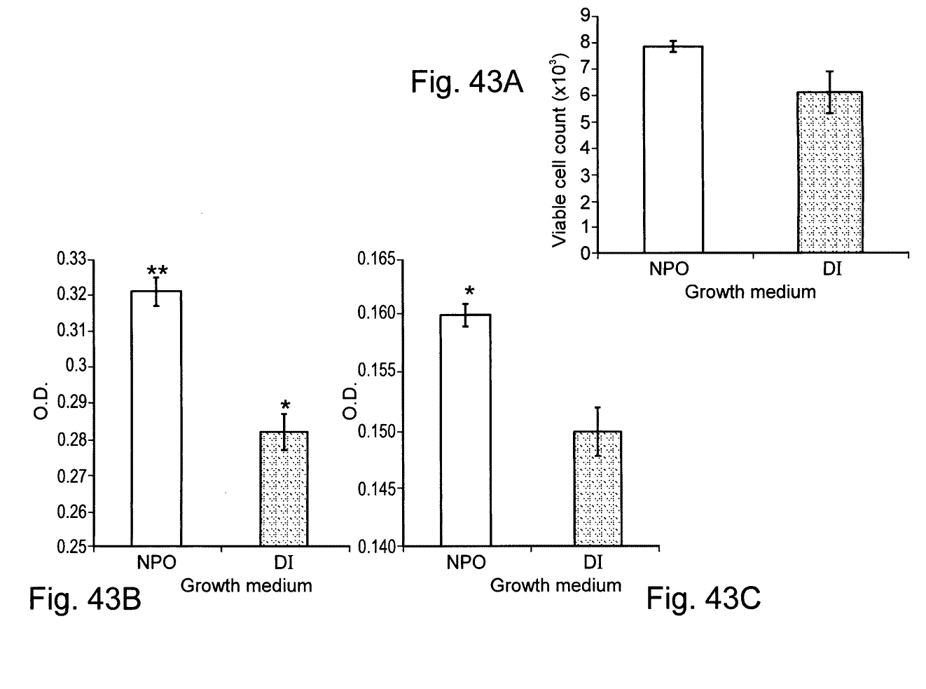

Firstly, the present inventors have shown that the liquid composition is capable of increasing the proliferation rate of cells cultured therein (FIG. 1, Example 3 and FIGS. 43A-C, Example 19).

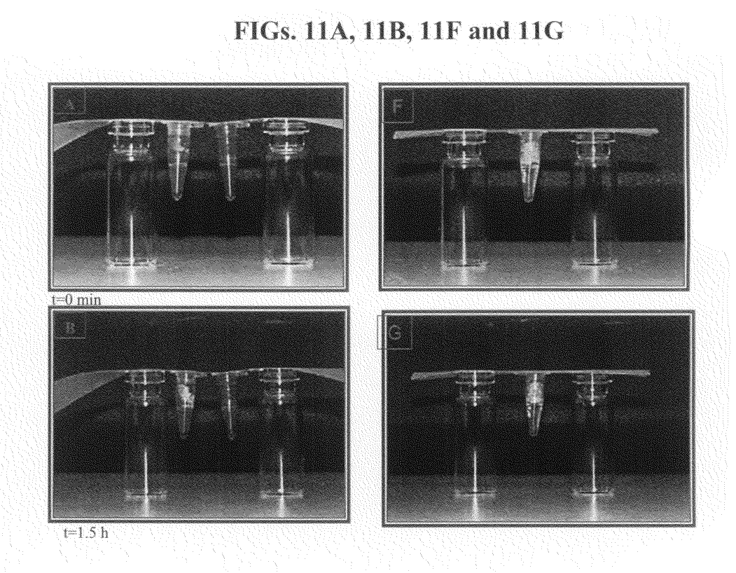

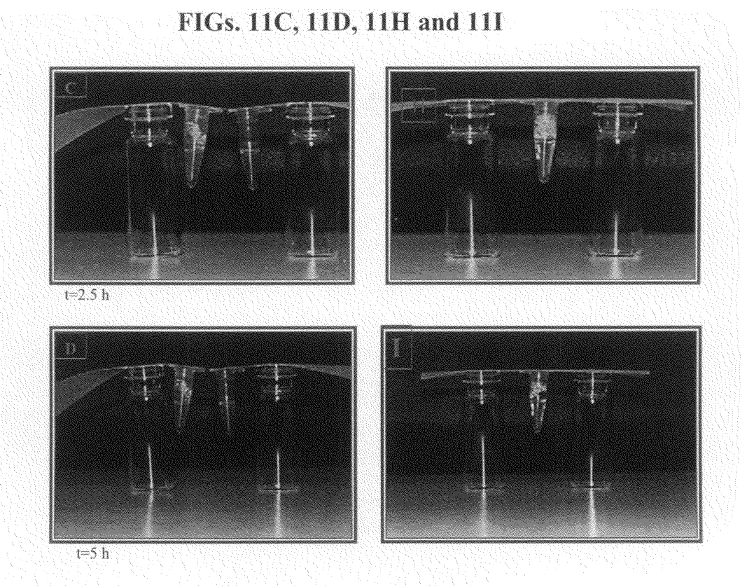

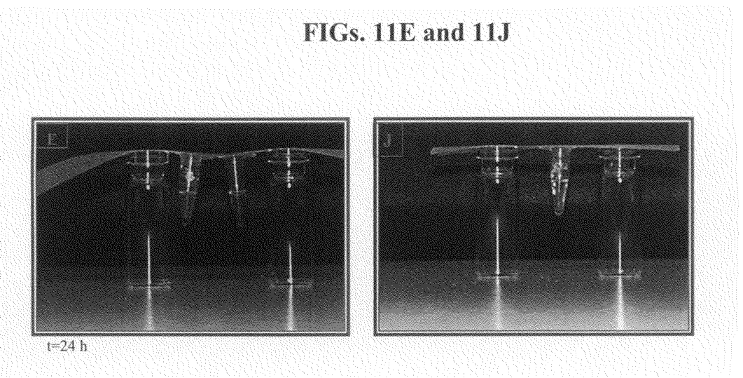

Secondly, the present inventors have shown that the liquid composition of the present invention enhances the solubility of agents (Examples 8-15, FIGS. 11-34 and Examples 18 and 21). This may be particularly relevant for enhancing the solubility of a water-insoluble agent that needs to be added to a culture medium.

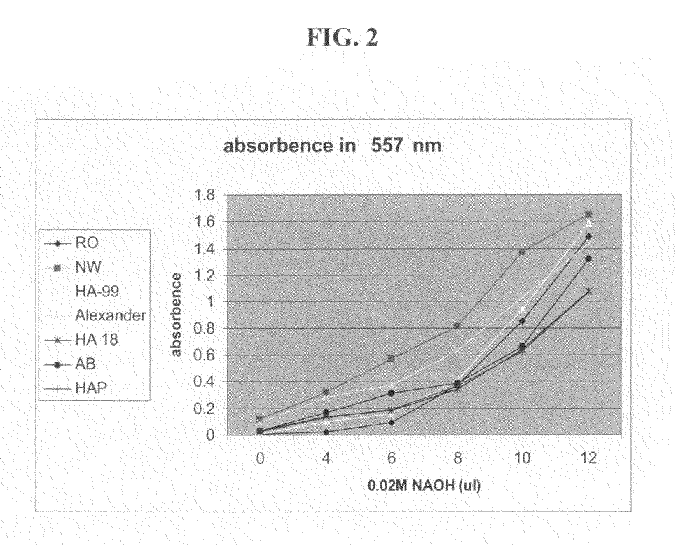

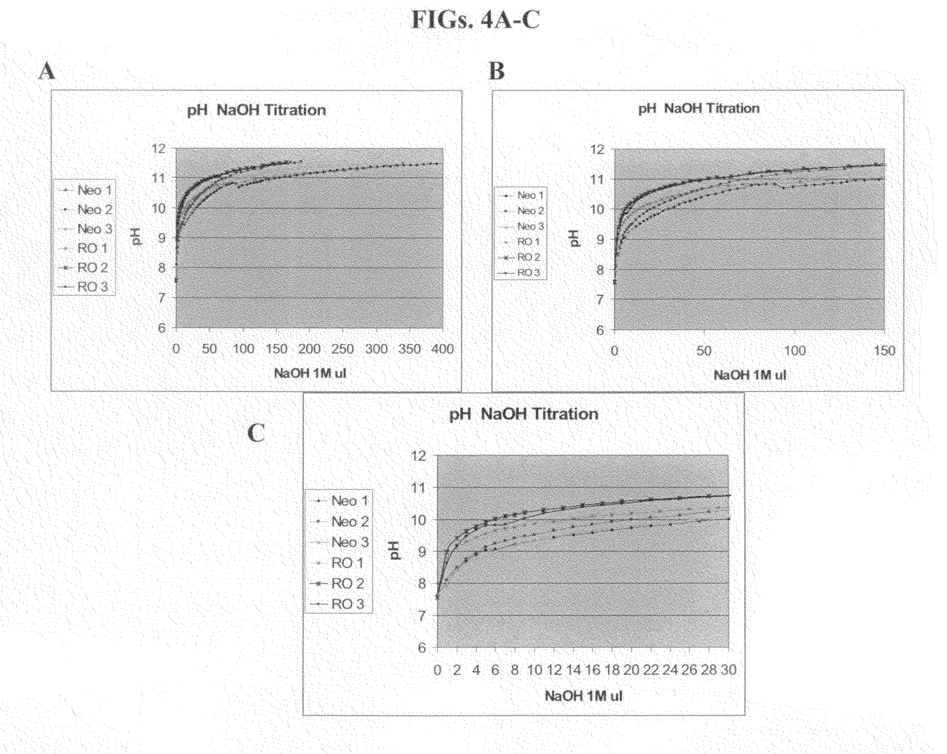

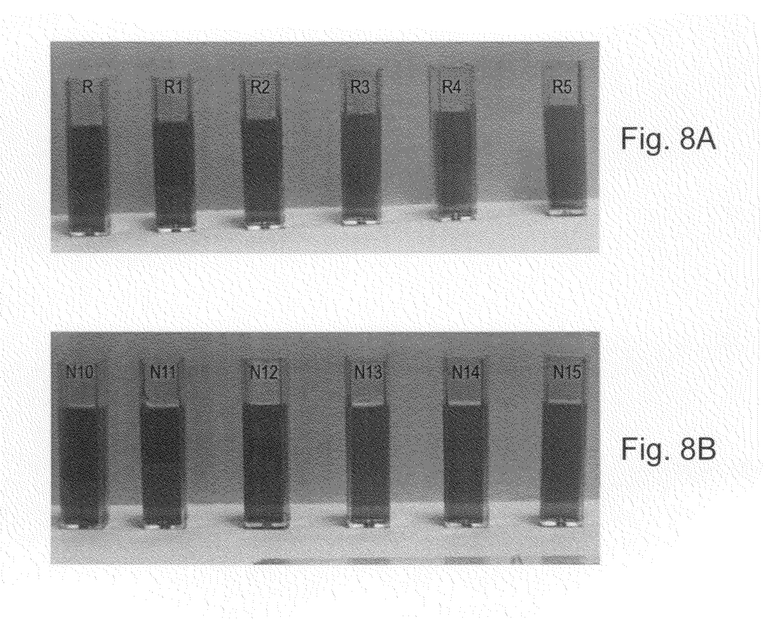

Thirdly, the present inventors have shown that the liquid composition of the present invention comprises an enhanced buffering capacity i.e. comprises a buffering capacity greater than water (Examples 4-7, FIGS. 2-10). This may be relevant for cells that are particulary pH sensitive.

As used herein, the phrase “buffering capacity” refers to the composition's ability to maintain a stable pH stable as acids or bases are added.

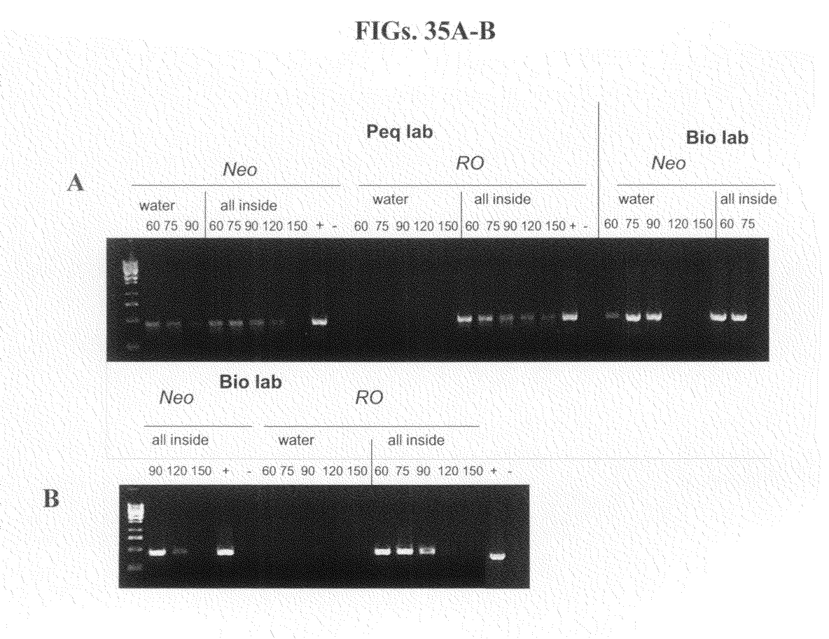

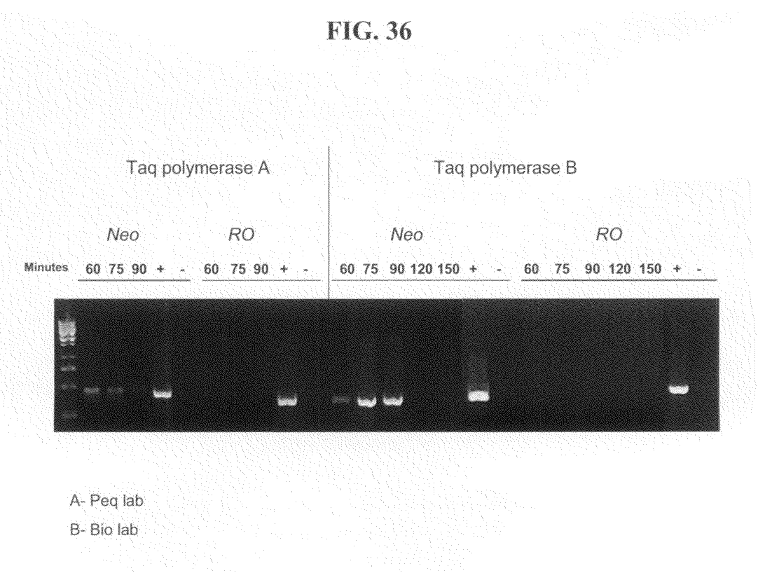

Lastly, the present inventors have shown that the liquid composition of the present invention is capable of stabilizing proteins (Examples 16-16, FIGS. 35-36). This may be particularly relevant if a non-stable peptide agent needs to be added to a culture medium or for stabilizing a cell secreted peptide agent.

It should be appreciated that according to this aspect of the present invention, the cells may be cultured for any purposes, such as, but not limited to for growth, maintenance and/or for cloning. In addition, it should be appreciated that the incubation time is not restricted in any way and cells may be cultured in the composition of the present invention for as long as required.

The composition of the present invention may be particularly useful for culturing cells which require autocrine secretion of factors which are typically present at low concentrations. For example, mesencymal stem cells were shown to secrete DKK1, which enhances proliferation. The ordered structure of the composition of the present invention may serve to effectively increase the DKK1 concentration thereby enhancing its growth.

The composition of the present invention may also be particularly useful for culturing cells which have a tendency to be non-stable. Examples of such cells include, but are not limited to hybridomas, cells which are being re-cultured following freezing and cells which are present at low concentrations.

The present inventors contemplate exchanging all or a part of the water content of any eukaryotic cell culture medium with the liquid composition of the present invention. Removal of the water content of the medium may be effected using techniques such as lyophilization, air-drying and oven-drying. Thus, the liquid portion of the culturing medium may comprise 5%, more preferably 10%, more preferably 20%, more preferably 40%, more preferably 60%, more preferably 80% and even more preferably 100% of the liquid composition of the present invention.

Many media are also commercially available as dried components. As such, the liquid composition of the present invention may be added without the prior need to remove the water component of the media.

Examples of eukaryotic cell culture media include DMEM, RPMI, Ames Media, CHO cell media, Ham's F-10 medium, Ham's F-12 medium, Leibovita L-15 medium, McCoy's medium, MEM Alpha Medium. Such media are widely available from Companies such as Sigma Aldrich and Invitrogen.

It will be appreciated that the medium may comprise other components such as growth factors, serum and antibiotics. Such components are commercially available e.g. from Sigma Aldrich and Invitrogen.

Preferably the liquid composition of the present invention is sterilized (e.g. by filter sterilization) prior to incubating the cells therein.

According to yet a further aspect of the present invention, there is provided an article-of-manufacture, which comprises the composition of the present invention as described hereinabove, being packaged in a packaging material and identified in print, in or on the packaging material for culturing eukaryotic cells, as described herein.

As mentioned hereinabove, the composition of the present invention may be manufactured as a ready-made culture medium. Accordingly, there is provided a cell culture medium comprising a eukaryotic cell culture medium and a liquid composition as described hereinabove.

According to another aspect of the present invention there is provided a method of dissolving or dispersing cephalosporin, comprising contacting the cephalosporin with nanostructures and liquid under conditions which allow dispersion or dissolving of the substance, wherein the nanostructures comprise a core material of a nanometric size enveloped by ordered fluid molecules of the liquid, the core material and the envelope of ordered fluid molecules being in a steady physical state.

The cephalosporin may be dissolved in a solvent prior or following addition of the liquid composition of the present invention in order to aid in the solubilizing process. It will be appreciated that the present invention contemplates the use of any solvent including polar, non-polar, organic, (such as ethanol or acetone) or non-organic to further increase the solubility of the substance.

The solvent may be removed (completely or partially) at any time during the solubilizing process so that the substance remains dissolved/dispersed in the liquid composition of the present invention. Methods of removing solvents are known in the art such as evaporation (i.e. by heating or applying pressure) or any other method.

As used herein the term “about” refers to ? 10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Haines, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Effect of Water Comprising Nanostructures on the Isolation of Human Hybridomas

The following experiments were performed in order to ascertain whether water comprising nanostructures affects the first stage of monoclonal antibody production—isolation of hybridomas.

Materials and Methods

Reagents for cell growth: RPMI 1640 was purchased in powder from Beit-HaEmek, Israel and reconstituted either in neowater™ (Decoop, Israel) or in control water, purified by reverse osmosis. Following reconstitution, sodium bicarbonate was added to the media according to the manufacturers' recommendation, and the pH was adjusted to 7.4. The culture media were supplemented with 10% fetal calf serum, L-glutamine (4 mM), penicillin (100 U/mL), streptomycin (0.1 mg/mL), MEM-vitamins (0.1 mM), non-essential amino acids (0.1 mM) and sodium pyruvate (1 mM)—all purchased from GIBCO BRL, Life Technologies. HCF was purchased from OriGen. All the supplements mentioned above were bought in a liquid, water-based form and diluted into the neowater™-based or control media. 8-Azaguanine, HT and HAT were purchased from Sigma and reconstituted from powder form with either neowater™-based or control RPMI.

Chemical reagents: Powdered PBS (GIBCO BRL, Life Technologies) was reconstituted with either neowater™ or control water. Flaked PEG-1500 (Sigma) was reconstituted with both forms of sterile PBS (50% w/v); the pH of the liquid PEG was adjusted to ˜7 and it was filter-sterilized. Hanks balanced salt solution was bought from Beit-HaEmek. Carbonate-bicarbonate buffer (0.05 M, pH=9.6) for ELISA plate-coating, OPD (used in 0.4 mg/mL) and phosphate-citrate buffer (0.05 M, pH=5.0) were bought from Sigma.

Antibodies: Goat anti-human IgM and HRP-conjugated goat anti-human IgM were purchased from Jackson ImmunoResearch. Standard human IgM was bought from Sigma.

Fusion: Human peripheral blood mononuclear cells (PBMC) and fusion partner (MFP-2) cells were washed 4 times in serum-free culture medium prior to mixing and pelleting. 300 μl of PEG-1500, pre-warmed to 37° C. was added to the cell mixture (10-200×10⁶ cells) and incubated for 3 minutes with constant shaking. PEG was then diluted out of the cell mixture with Hanks balanced salt solution and complete RPMI. Fetal calf serum (10%) and HT (×2) were added to the resultant cell suspension. The hybridomas that were generated during this process were cultured in a 96-well plate in complete RPMI with HAT selection. The screening of the supernatants for antibodies began when hybridoma cells occupied approximately ¼ of the well.

Sandwich ELISA: A sandwich ELISA was used to screen hybridoma supernatants for IgM. Briefly, a capturing antibody (goat anti-human IgM) was prepared in a carbonate/bicarbonate buffer and applied to the 96-well plate in a concentration of 100 ng/100 μl/well. The plate was then incubated overnight at 4° C. All the following steps were performed at room temperature. After 1 hour of blocking with 0.3% dry milk in PBS, the supernatants from the hybridomas were added for a duration of 1.5 hours. Human serum diluted 1:500 in PBS was used as a positive control. Hybridoma growth medium was used as a negative control. The secondary antibody (HRP-conjugated goat anti-human IgM) was prepared in blocking solution at a concentration of 1:5000 and incubated for 1 hour. To produce a colorimetric reaction, the plates were incubated with OPD in phosphate-citrate buffer, containing 0.03% H₂O₂. The color reaction was stopped with 10% Hydrochloric acid after 15 minutes. The reading and the recording of the reaction were performed on the Multiscan-Ascent using the 492 nm wavelength filter.

Results

Two sets of identical experiments were performed, the first with all neowater™-based reagents (except for the addition of liquid supplements) and the second with reagents made in standard reverse osmosis water (herein control water). The use of neowater™ in the described experiments was started at the point of cell growth i.e. two populations of MFP-2 cells were plated at equal densities: one in the neowater™-based complete RPMI and the other in control water-based complete RPMI. At this stage MFP-2 were grown in the presence of 8-Azaguanine, to select out HAT-resistant cells. After a week of growth, the two fusion experiments were performed with an equal number of lymphocytes and MFP-2 cells. Each fusion was plated on to 8 plates of 96-wells. Approximately 20 days later, the hybridomas from both fusions were tested for their ability to produce IgM. The numbers of IgM-positive clones found in each plate are presented in Table 1 below.

TABLE 1

Number of
IgM-positive wells in
plate

| Plate number | control | neowater ™ |
|---|---|---|
| 1 | 61 | 88 |
| 2 | 14 | 88 |
| 3 | 22 | 80 |
| 4 | 8 | 87 |
| 5 | 32 | 70 |
| 6 | 66 | 85 |
| 7 | 1 | 88 |
| 8 | 0 | 87 |
| Total positive wells | 204 | 673 |
| Mean value | 25 | 84 |
| Number of wells with concentration | 5 | 18 |
| higher than control | | |
| Percent of hybridomas with IgM | 2.5% | 2.7% |
| concentration higher then control | | |

A statistically significant difference was found between the average numbers of IgM-positive wells in control and in neowater™ (Unpaired two-tailed t-test for control vs. neowater: p<0.001). In addition, as seen in the table above, the number of hybridomas per plate is relatively constant, indicating that neowater™ enables more consistency in the production of hybridomas, likely a result of its stabilizing influence. Therefore the entire process of creating and isolating stable hybridoma clones that secrete human monoclonal antibodies is greatly enhanced in neowater™.

The amount of IgM-positive wells in control and neowater™, (where the measured concentrations were higher than the diluted serum) was also measured. As illustrated in Table 1, no statistically significant difference was detected using χ² test. This strongly suggests that neowater™ affects the formation and stabilization of the hybridomas and does not play as great a role in the level of secretion.

The kinetics of secretion of antibodies in neowater™ based media following hybridoma formation was analyzed. It has been shown that neowater™ does increase secretion, although it may do so by stabilizing the hybridomas thereby enabling a higher overall secretion rate, rather than by effecting the secretory machinery of the cell.

Since the difference in the numbers of IgM-producing hybridomas between control and neowater™ could be attributed to ELISA measurements being more precise in a neowater-milieu, the following experiment was performed: Three calibration curves were prepared where a standard human IgM was diluted in the following: 1) Tris buffer, 2) control growth medium (complete RPMI), 3) neowater™ growth medium (complete RPMI). The results of this experiment are presented in Table 2 hereinbelow.

TABLE 2

IgM conc. (μg/mL)

| | 6.25 | 12.5 | 25 | 50 |
|---|---|---|---|---|
| Tris buffer | 0.382 | 0.504 | 0.932 | 1.327 |
| Control medium | 0.749 | 0.852 | 0.908 | 0.980 |
| neowater | 0.628 | 0.800 | 0.88 | 0.948 |
| medium | | | | |

As table 2 shows, both the control and the neowater™ media somewhat distort the values of optical density compared to Tris buffer (which according to the manufacturers recommendation the standard IgM should be calibrated). However no statistically significant difference was found between the control and neowater™ values of optical density in the tested range.

### Example 2

### Effect of Water Comprising Nanostructures on the Cloning of Human Hybridomas

The next step in monoclonal antibody production following isolation of a relevant hybridoma is stabilizing it by cloning. To test whether water comprising nanostructures can interfere with the clonabilty of hybridomas the following experiment was conducted.

Materials and Methods

Cloning: Cloning of hybridomas was performed according to standard protocols. Briefly, a limited number (approx. 10⁴) of cells were serially diluted across the top row of a 96 well dish and then the contents of the first row were serially diluted down the remaining 8 rows. In this way, wells toward the right bottom of the plate tended to have single cells.

Screening for IgM content: A sandwich ELISA was used to screen hybridoma supernatants for IgM. Briefly, a capturing antibody (goat anti-human IgM) was prepared in a carbonate bicarbonate buffer and applied on a 96-well plate in a concentration of 100 ng/100 μL/well. The plate was then incubated overnight at +4° C. All the following steps were performed at room temperature. Following 1 hour of blocking with 0.3% dry milk in PBS, the supernatants from the hybridomas were applied for 1.5 hours. Human serum diluted 1:500 in PBS was used as a positive control. For a background and as a negative control hybridoma growth medium was used. The secondary antibody (HRP-conjugated goat anti-human IgM) was prepared in blocking solution at a concentration of 1:5000 and incubated for 1 hour. To produce calorimetric reaction, the plates were incubated with OPD in phosphate-citrate buffer, containing 0.03% H₂O₂. The color reaction was stopped with 10% Hydrochloric acid after 15 minutes. The reading and the recording of the reaction were performed on the Multiscan-Ascent using the 492 nm wavelength filter.

Results

Three subclone plates were prepared from the same positive parent-well in the following manner: plate I was subcloned in neowater™ media without the addition of HCF; plate II was subcloned in control media with the addition of HCF; plate III was subcloned in neowater™ media with HCF. The plates were followed up microscopically for two weeks, after which the cell density in the wells was high enough to produce measurable amounts of antibodies. The supernatants of the three plates were then tested for their IgM content. The results summarizing this experiment are presented in Table 3 hereinbelow.

TABLE 3

| | Plate I | Plate II | Plate III |
|---|---|---|---|
| Median | 0.217 | 0.205 | 0.264 |
| Average | 0.319 | 0.341 | 0.318 |
| St. Dev. | 0.285 | 0.310 | 0.253 |
| Number of IgM-positive | 28 | 34 | 32 |
| wells | | | |

*Cloning protocol: Plate I - in neowater ™ complete RPMI; Plate II in control complete RPMI + HCF (10%); Plate III in neowater ™ complete RPMI + HCF (10%).

No statistically significant difference was found among the frequency of IgM-producing clones or the distributions of antibody amounts produced in each plate indicating that the hybridomas clone as well in neowater™ based media as in control media with HCF. In control media, hybridomas do not clone without the addition of HCF. This suggests that neowater™ based media creates an environment that enhances clonability of unstable hybridomas. This notion is also borne out by the enhanced frequency of hybridoma recovery following fusion in neowater™ based reagents and growth in neowater™ based media.

Discussion and Conclusions

The results of the experiments described herein indicate that neowater™ improves the fusion process, whether by means of elevating the physical cell fusion efficiency or by means of stabilizing the hybridomas created in the process of fusion. Either way the yields of a fusion prepared in neowater™ were significantly higher than in the control (p<0.001 Table 1). Also, neowater™ probably does not interfere in the mechanisms of antibody production or secretion, since the percent of high-yield hybridomas and the distribution of antibodies concentrations do not significantly differ between control and neowater™ tests (Table 1).

However, the cloning experiment revealed important evidence supporting the hypothesis that neowater™ may have a stabilizing effect on new clones. Cloning without the HCF in most cases does not lead to successful and stable clones. The role of this reagent, which in fact is a macrophage-conditioned medium, is to support single-cell growth. Without the factors received by hybridomas from the HCF, they mostly die or manage to multiply but loose their capacity to produce antibodies. The fact that viable, antibody-secreting hybridomas were obtained while cloning in neowater™ without HCF is a valuable finding in itself. Moreover, these clones are equal in their productivity and frequency when statistically compared to clones that were established in HCF-cloning.

Other observations also support the hypothesis of neowater™'s stabilizing effect. For example, cell populations that failed to recover after thawing into control medium did slowly recover in neowater™-based medium. Productive clones that were generated and grown in a neowater™-environment stopped secreting antibodies when the medium was changed to control for a day.

### Example 3

### Effect of the Liquid Composition Comprising Nanostructures on Proliferation

The following experiment was performed on human Mesenchymal cells to ascertain if the liquid composition comprising nanostructures effects cell proliferation.

Materials and Methods

Proliferation of human mesenchymal stem cells were examined in mediums based on RO water or Neowater™.

Preparation of medium: 250 ml of MEM alpha medium were prepared by addition of 2.5 g of MEM and 0.55 g of Na HCO3 either to RO water of Neowater™.

Cell culture: The cells were maintained in MEM α supplemented with 20% fetal calf serum, 100 u/ml penicillin and 1 mg/ml streptomycin (Colter et al., 2001, PNAS 98:7841-7845). Cells were counted and diluted to the concentration of 500 cells per ml, in 2 types of MEM α medium; one based on RO water, and the other based on Neowater™. Cells were grown in a 96 well plate, 100 μl medium with 50 cells in each well. After 8 days, cell proliferation was estimated by a crystal violet viability assay. The dye in this assays, stains DNA. Upon solubilization, the amount of the dye taken up by the monolayer can be quantitated in a plate reader, at 590 nm.

Results

The results of the crystal violet viability assay are summarized hereinbelow in Table 4 and FIG. 1.

TABLE 4

| | | | | | | |
|---|---|---|---|---|---|---|
| | 0.032355 | 0.013255 | 0.065955 | 0.047855 | 0.054855 | |
| | 0.011455 | 0.014955 | 0.035255 | 0.068055 | 0.073255 | Neowater |
| | 0.051955 | 0.070455 | 0.053155 | 0.073955 | 0.045355 | |
| | 0.029055 | 0.030555 | 0.037055 | 0.023455 | 0.041955 | |
| | 0.005555 | 0.009155 | 0.018355 | 0.005455 | 0.072455 | |
| | 0.026955 | 0.008255 | 0.012055 | 0.007155 | 0.046055 | |
| | 0.010555 | 0.017555 | 0.030155 | 0.002055 | 0.023255 | RO |
| | 0.029955 | 0.001955 | 0.020855 | 0.025255 | 0.022955 | |
| T test | 0.000238 | | | | | |

Conclusion

The liquid composition of the present invention increases the proliferation of cells.

### Example 4

### Buffering Capacity of the Composition Comprising Nanostructures

The effect of the composition comprising nanostructures on buffering capacity was examined.

Materials and Methods

Phenol red solution (20 mg/25 ml) was prepared. 290 μl was added to 13 ml RO water or various batches of water comprising nanostructures (Neowater™—Do-Coop technologies, Israel). It was noted that each water had a different starting pH, but all of them were acidic, due to their yellow or light orange color after phenol red solution was added. 2.5 ml of each water+phenol red solution were added to a cuvette. Increasing volumes of Sodium hydroxide were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Results

Table 5 summarizes the absorbance at 557 nm of each water solution following sodium hydroxide titration.

TABLE 5

| | | | | | | | μl of 0.02 |
|---|---|---|---|---|---|---|---|
| NW | NW 2 | | | NW 5 | | | M sodium |
| 1 | AB | NW 3 | NW 4 | HA- | NW | | hydroxide |
| HAP | 1-2-3 | HA 18 | Alexander | 99-X | 6 | RO | added |
| 0.026 | 0.033 | 0.028 | 0.093 | 0.011 | 0.118 | 0.011 | 0 |
| 0.132 | 0.17 | 0.14 | 0.284 | 0.095 | 0.318 | 0.022 | 4 |
| 0.192 | 0.308 | 0.185 | 0.375 | 0.158 | 0.571 | 0.091 | 6 |
| 0.367 | 0.391 | 0.34 | 0.627 | 0.408 | 0.811 | 0.375 | 8 |
| 0.621 | 0.661 | 0.635 | 1.036 | 0.945 | 1.373 | 0.851 | 10 |
| 1.074 | 1.321 | 1.076 | 1.433 | 1.584 | 1.659 | 1.491 | 12 |

As illustrated in FIG. 2 and Table 5, RO water shows a greater change in pH when adding Sodium hydroxide. It has a slight buffering effect, but when absorbance reaches 0.09 A, the buffering effect “breaks”, and pH change is greater following addition of more Sodium hydroxide. HA-99 water is similar to RO. NW (#150905-106) (Neowater™), AB water Alexander (AB 1-22-1 HA Alexander) has some buffering effect. HAP and HA-18 shows even greater buffering effect than Neowater™.

In summary, from this experiment, all new water types comprising nanostructures tested (HAP, AB 1-2-3, HA-18, Alexander) shows similar characters to Neowater™, except HA-99-X.

### Example 5

### Buffering Capacity of the Liquid Composition Comprising Nanostructures

The effect of the liquid composition comprising nanostructures on buffering capacity was examined.

Materials and Methods

Sodium hydroxide and Hydrochloric acid were added to either 50 ml of RO water or water comprising nanostructures (Neowater™—Do-Coop technologies, Israel) and the pH was measured. The experiment was performed in triplicate. In all, 3 experiments were performed.

Sodium hydroxide titration: −1 μl to 15 μl of 1M Sodium hydroxide was added.

Hydrochloric acid titration: −1 μl to 15 μl of 1M Hydrochloric acid was added.

Results

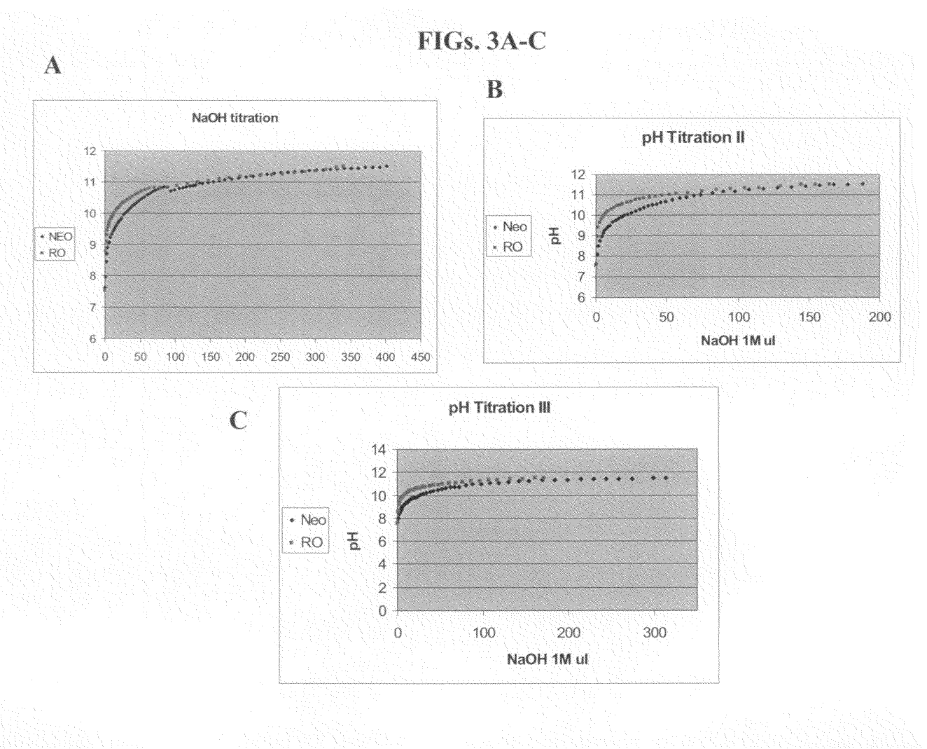

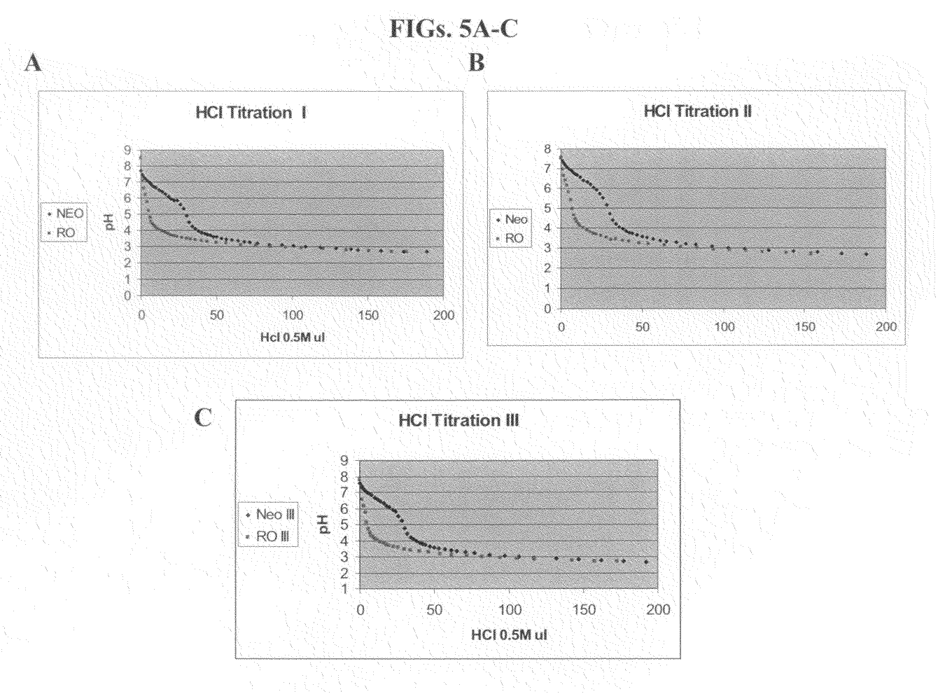

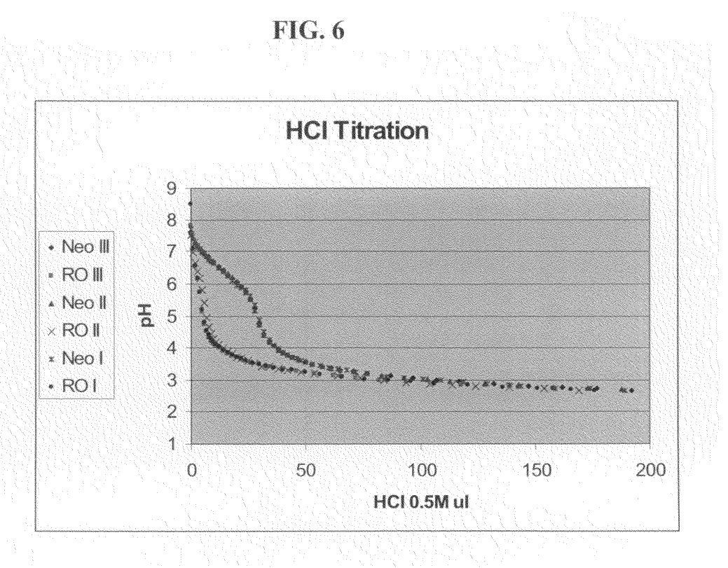

The results for the Sodium hydroxide titration are illustrated in FIGS. 3A-C and 4A-C. The results for the Hydrochloric acid titration are illustrated in FIGS. 5A-C and FIG. 6.

The water comprising nanostructures has buffering capacities since it requires greater amounts of Sodium hydroxide in order to reach the same pH level that is needed for RO water. This characterization is more significant in the pH range of −7.6-10.5. In addition, the water comprising nanostructures requires greater amounts of Hydrochloric acid in order to reach the same pH level that is needed for RO water. This effect is higher in the acidic pH range, than the alkali range. For example: when adding 10 μl Sodium hydroxide 1M (in a total sum) the pH of RO increased from 7.56 to 10.3. The pH of the water comprising nanostructures increased from 7.62 to 9.33. When adding 10 μl Hydrochloric acid 0.5M (in a total sum) the pH of RO decreased from 7.52 to 4.31 The pH of water comprising nanostructures decreased from 7.71 to 6.65. This characterization is more significant in the pH range of −7.7-3.

### Example 6

### Buffering Capacity of the Liquid Composition Comprising Nanostructures

The effect of the liquid composition comprising nanostructures on buffering capacity was examined.

Materials and Methods

Phenol red solution (20 mg/25 ml) was prepared. 1 ml was added to 45 ml RO water or water comprising nanostructures (Neowater™—Do-Coop technologies, Israel). pH was measured and titrated if required. 3 ml of each water+phenol red solution were added to a cuvette. Increasing volumes of Sodium hydroxide or Hydrochloric acid were added to each cuvette, and absorption spectrum was read in a spectrophotometer. Acidic solutions give a peak at 430 nm, and alkaline solutions give a peak at 557 nm. Range of wavelength is 200-800 nm, but the graph refers to a wavelength of 557 nm alone, in relation to addition of 0.02M Sodium hydroxide.

Hydrochloric acid Titration:
RO: 45 ml pH 5.8
   1 ml phenol red and 5 μl Sodium hydroxide 1M was added, new pH=7.85 Neowater™ (#150905-106): 45 ml pH 6.3
   1 ml phenol red and 4 μl Sodium hydroxide 1M was added, new pH=7.19

Sodium Hydroxide Titration:
I. RO: 45 ml pH 5.78
   1 ml phenol red, 6 μl Hydrochloric acid 0.25M and 4 μl Sodium hydroxide 0.5M was added, new pH=4.43
Neowater™ (#150604-109): 45 ml pH 8.8
   1 ml phenol red and 45 μl Hydrochloric acid 0.25M was added, new pH=4.43
II. RO: 45 ml pH 5.78
   1 ml phenol red and 5 μl Sodium hydroxide 0.5M was added, new pH=6.46 Neowater™ (#120104-107): 45 ml pH 8.68
   1 ml phenol red and 5 μl Hydrochloric acid 0.5M was added, new pH=6.91

Results

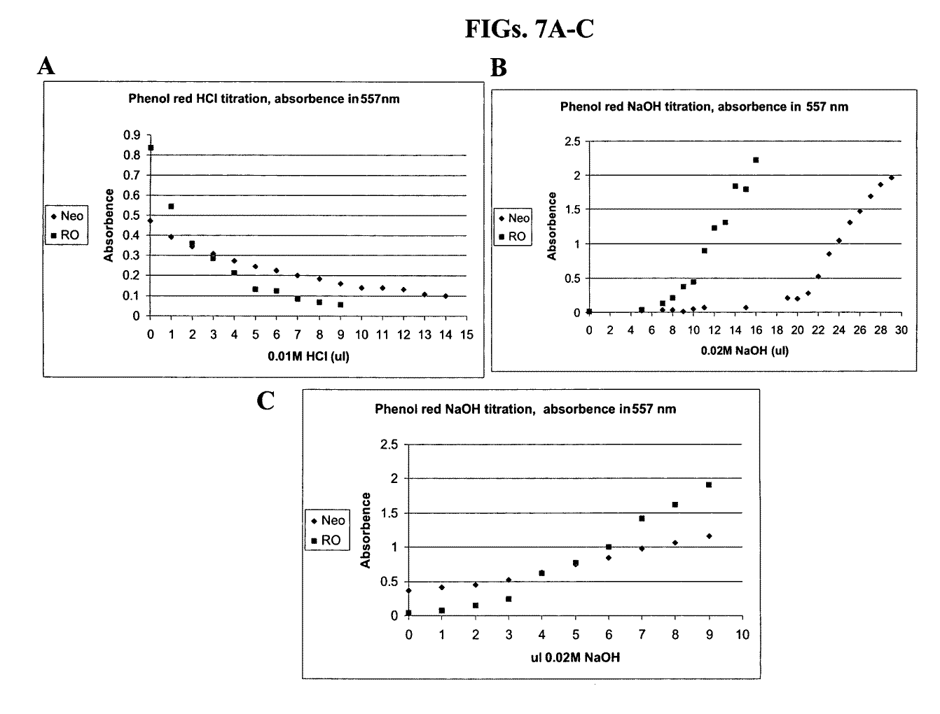

As illustrated in FIGS. 7A-C and 8A-B, the buffering capacity of water comprising nanostructures was higher than the buffering capacity of RO water.

### Example 7

### Buffering Capacity of RF Water

The effect of the RF water on buffering capacity was examined.

Materials and Methods

A few μl drops of Sodium hydroxide 1M were added to raise the pH of 150 ml of RO water (pH=5.8). 50 ml of this water was aliquoted into three bottles.

Three treatments were done:

Bottle 1: no treatment (RO water)

Bottle 2: RO water radiated for 30 minutes with 30 W. The bottle was left to stand on a bench for 10 minutes, before starting the titration (RF water).

Bottle 3: RF water subjected to a second radiation when pH reached 5. After the radiation, the bottle was left to stand on a bench for 10 minutes, before continuing the titration.

Titration was performed by the addition of 1 μl0.5M Hydrochloric acid to 50 ml water. The titration was finished when the pH value reached below 4.2.

The experiment was performed in triplicates.

Results

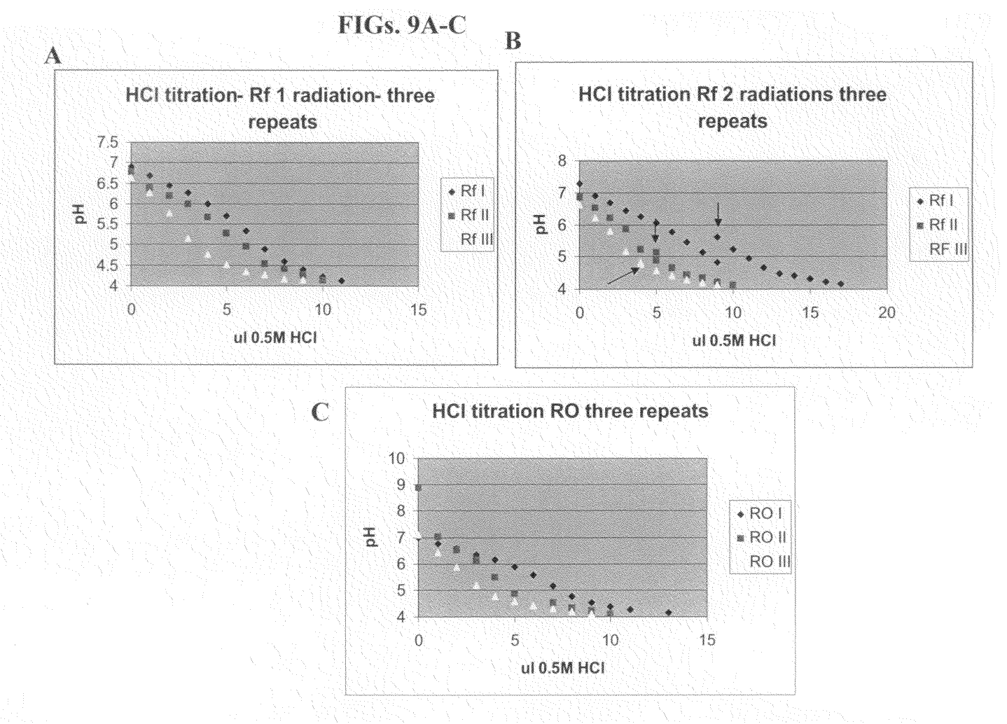

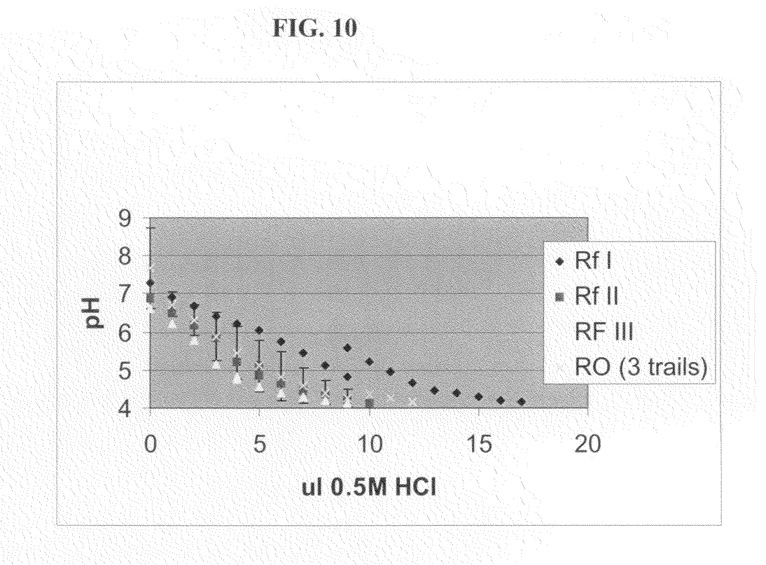

As can be seen from FIGS. 9A-C and FIG. 10, RF water and RF2 water comprise buffering properties similar to those of the carrier composition comprising nanostructures.

### Example 8

### Solvent Capability of the Liquid Composition Comprising Nanostructures

The following experiments were performed in order to ascertain whether the liquid composition comprising nanostructures was capable of dissolving two materials both of which are known not to dissolve in water at a concentration of 1 mg/ml.

A. Dissolving in Ethanol/(Neowater™—Do-Coop Technologies, Israel) Based Solutions

Materials and Methods

Five attempts were made at dissolving the powders in various compositions. The compositions were as follows:
A. 10 mg powder (red/white)+990 μl Neowater™.
B. 10 mg powder (red/white)+990 μl Neowater™ (dehydrated for 90 min).
C. 10 mg powder (red/white)+495 μl Neowater™+495 μl EtOH (50%-50%).
D. 10 mg powder (red/white)+900 μl Neowater™+90 μl EtOH (90%-10%).
E. 10 mg powder (red/white)+820 μl Neowater™+170 μl EtOH (80%-20%).
   The tubes were vortexed and heated to 60° C. for 1 hour.

Results
1. The white powder did not dissolve, in all five test tubes.
2. The red powder did dissolve however; it did sediment after a while. It appeared as if test tube C dissolved the powder better because the color changed to slightly yellow.

B. Dissolving in Ethanol/(Neowater™—Do-Coop Technologies, Israel) Based Solutions Following Crushing

Materials and Methods

Following crushing, the red powder was dissolved in 4 compositions:
A. ½ mg red powder+49.5 μl RO.
B. ½ mg red powder+49.5 μl Neowater™.
C. ½ mg red powder+9.9 μl EtOH→39.65 μl Neowater™ (20%-80%).
D. ½ mg red powder+24.75 μl EtOH→24.75 μl Neowater™ (50%-50%).
Total reaction volume: 50 μl.
   The tubes were vortexed and heated to 60° C. for 1 hour.

Results

Following crushing only 20% of ethanol was required in combination with the Neowater™ to dissolve the red powder.

C. Dissolving in Ethanol/(Neowater™—Do-Coop Technologies, Israel) Solutions Following Extensive Crushing

Materials and Methods

Two crushing protocols were performed, the first on the powder alone (vial 1) and the second on the powder dispersed in 100 μl Neowater™ (1%) (vial 2).

The two compositions were placed in two vials on a stirrer to crush the material overnight:

15 hours later, 100 μl of Neowater™ was added to 1 mg of the red powder (vial no. 1) by titration of 10 μl every few minutes.

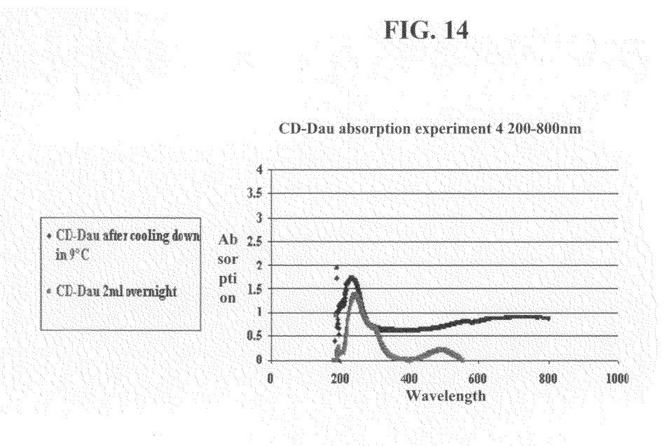

Changes were monitored by taking photographs of the test tubes between 0-24 hours (FIGS. 14F-J).

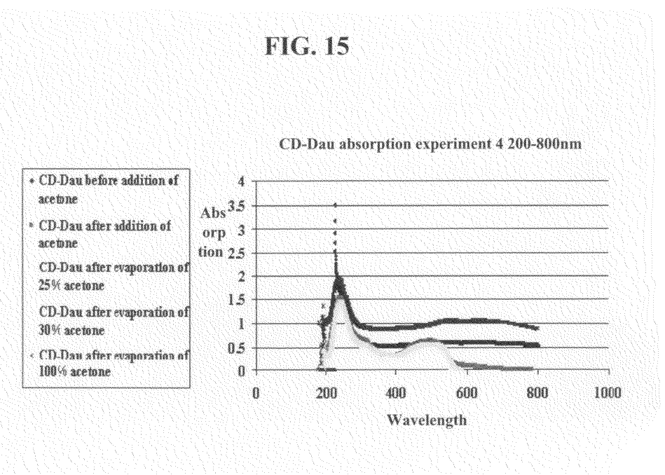

As a comparison, two tubes were observed one of which comprised the red powder dispersed in 990 μl Neowater™ (dehydrated for 90 min)—1% solution, the other dispersed in a solution comprising 50% ethanol/50% Neowater™)—1% solution. The tubes were heated at 60° C. for 1 hour. The tubes are illustrated in FIGS. 14A-E. Following the 24 hour period, 2 μl from each solution was taken and its absorbance was measured in a nanodrop (FIGS. 15A-C)

Results

FIGS. 11A-J illustrate that following extensive crushing, it is possible to dissolve the red material, as the material remains stable for 24 hours and does not sink. FIGS. 11A-E however, show the material changing color as time proceeds (not stable).

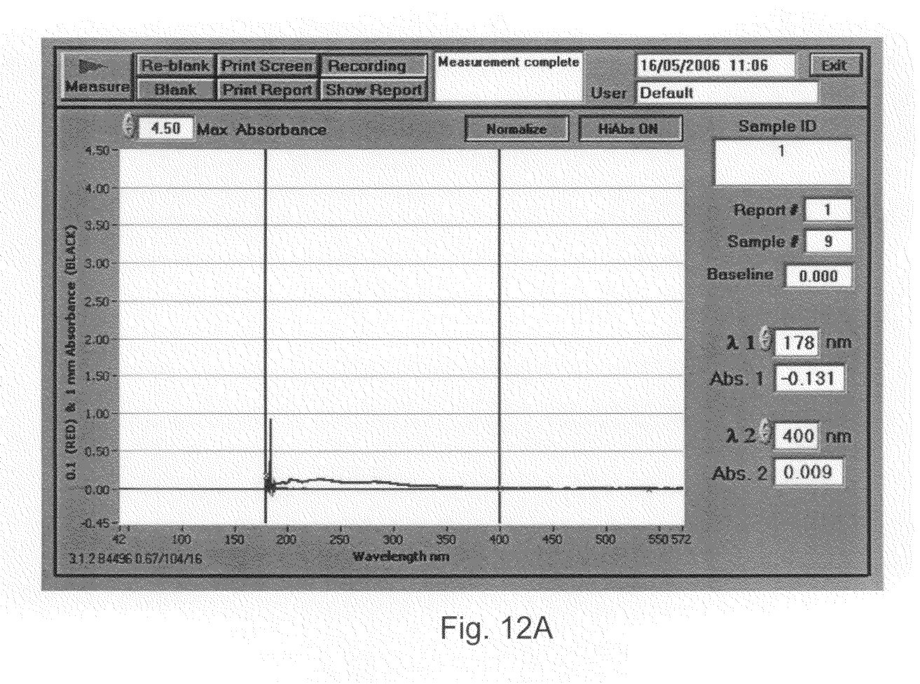

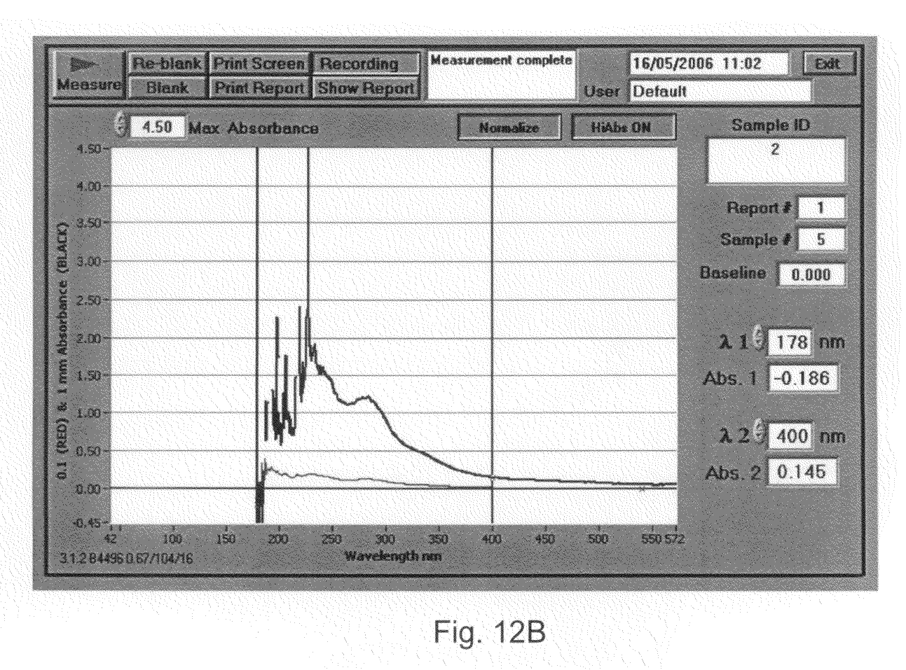

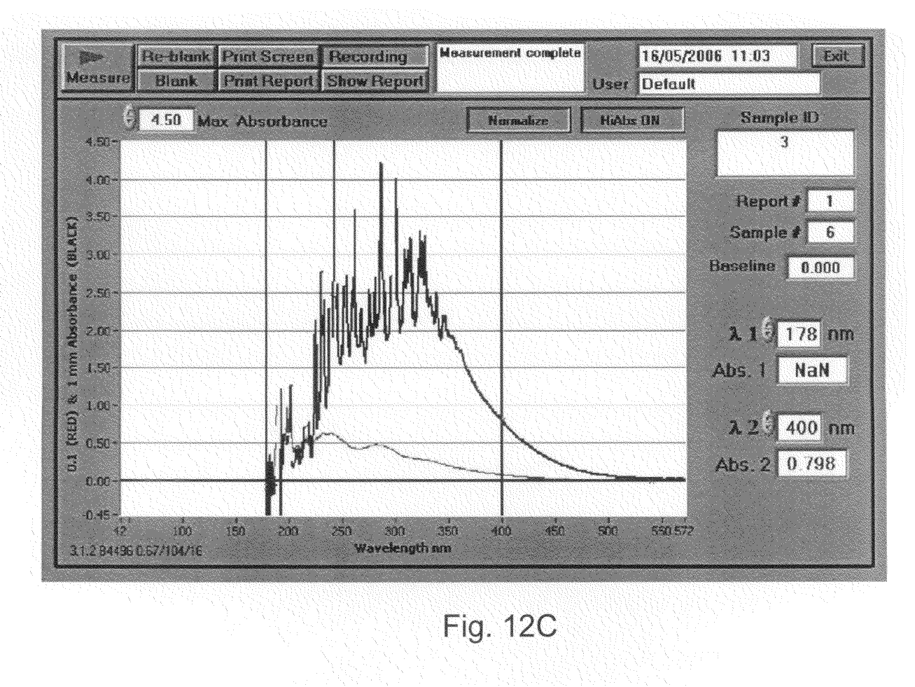

Vial 1 almost didn't absorb (FIG. 12A); solution B absorbance peak was between 220-270 nm (FIG. 12B) with a shift to the left (220 nm) and Solution C absorbance peak was between 250-330 nm (FIG. 12C).

Conclusions

Crushing the red material caused the material to disperse in Neowater™. The dispersion remained over 24 hours. Maintenance of the material in glass vials kept the solution stable 72 h later, both in 100% dehydrated Neowaterrm and in EtOH-Neowater™ (50% -50%).

### Example 9

### Capability of the Liquid Composition Comprising Nanostructures to Dissolve Daidzein, Daunrubicine and T-Boc Derivative

The following experiments were performed in order to ascertain whether the liquid composition comprising nanostructures was capable of dissolving three materials—Daidzein—daunomycin conjugate (CD-Dau); Daunrubicine (Cerubidine hydrochloride); t-boc derivative of daidzein (tboc-Daid), all of which are known not to dissolve in water.

Materials and Methods

A. Solubilizing CD-Dau—Part 1:

Required concentration: 3 mg/ml Neowater.
Properties: The material dissolves in DMSO, acetone, acetonitrile.
Properties: The material dissolves in EtOH.

5 different glass vials were prepared:
1. 5 mg CD-Dau+1.2 ml Neowater™.
2. 1.8 mg CD-Dau+600 μl acetone.
3. 1.8 mg CD-Dau+150 μl acetone+450 μl Neowater™ (25% acetone).
4. 1.8 mg CD-Dau+600 μl 10% *PEG (Polyethylene Glycol).
5. 1.8 mg CD-Dau+600 μl acetone+600 μl Neowater™.

The samples were vortexed and spectrophotometer measurements were performed on vials #1, 4 and 5

The vials were left opened in order to evaporate the acetone (vials #2, 3, and 5).

Results

Vial #1 (100% Neowater): CD-Dau sedimented after a few hours.

Vial #2 (100% acetone): CD-Dau was suspended inside the acetone, although 48 hours later the material sedimented partially because the acetone dissolved the material.

Vial #3 (25% acetone): CD-Dau didn't dissolve very well and the material floated inside the solution (the solution appeared cloudy).

Vial #4 (10% PEG+Neowater): CD-Dau dissolved better than the CD-Dau in vial #1, however it didn't dissolve as well as with a mixture with 100% acetone.

Vial #5: CD-Dau was suspended first inside the acetone and after it dissolved completely Neowater™ was added in order to exchange the acetone. At first acetone dissolved the material in spite of Neowater™'s presence. However, as the acetone evaporated the material partially sediment to the bottom of the vial. (The material however remained suspended.

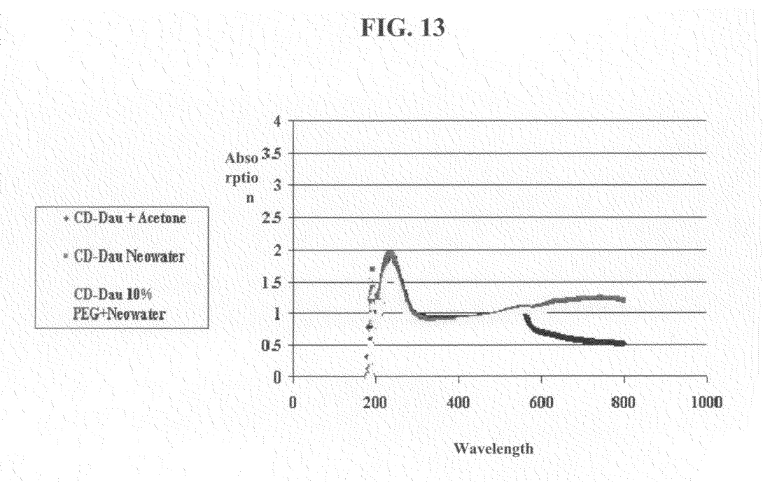

Spectrophotometer measurements (FIG. 13) illustrate the behavior of the material both in the presence and absence of acetone. With acetone there are two peaks in comparison to the material that is suspended with water or with 10% PEG, which in both cases display only one peak.

B. Solubilizing CD-Dau—Part 2:

As soon as the acetone was evaporated from solutions #2, 4 and 5, the material sedimented slightly and an additional amount of acetone was added to the vials. This protocol enables the dissolving of the material in the presence of acetone and Neowater™ while at the same time enabling the subsequent evaporation of acetone from the solution (this procedure was performed twice). Following the second cycle the liquid phase was removed from the vile and additional amount of acetone was added to the sediment material. Once the sediment material dissolved it was merged with the liquid phase removed previously. The merged solution was evaporated again. The solution from vial #1 was removed since the material did not dissolve at all and instead 1.2 ml of acetone was added to the sediment to dissolve the material. Later 1.2 ml of 10% PEG+Neowater™ were added also and after some time the acetone was evaporated from the solution. Finalizing these procedures, the vials were merged to one vial (total volume of 3 ml). On top of this final volume 3 ml of acetone were added in order to dissolve the material and to receive a lucid liquefied solution, which was then evaporated again at 50° C. The solution didn't reach equilibrium due to the fact that once reaching such status the solution would have been separated. By avoiding equilibrium, the material hydration status was maintained and kept as liquid. After the solvent evaporated the material was transferred to a clean vial and was closed under vacuum conditions.

C Solubilizing CD-Dau—Part 3:

Another 3 ml of the material (total volume of 6 ml) was generated with the addition of 2 ml of acetone-dissolved material and 1 ml of the remaining material left from the previous experiments.

1.9 ml Neowater™ was added to the vial that contained acetone.

100 μl acetone+100 μl Neowater™ were added to the remaining material.

Evaporation was performed on a hot plate adjusted to 50° C.

This procedure was repeated 3 times (addition of acetone and its evaporation) until the solution was stable.

The two vials were merged together.

Following the combining of these two solutions, the materials sedimented slightly. Acetone was added and evaporation of the solvent was repeated.

Before mixing the vials (3 ml+2 ml) the first solution prepared in the experiment as described in part 2, hereinabove was incubated at 9° C. over night so as to ensure the solution reached and maintained equilibrium. By doing so, the already dissolved material should not sediment. The following morning the solution's absorption was established and a different graph was obtained (FIG. 14). Following merging of the two vials, absorption measurements were performed again because the material sediment slightly. As a result of the partial sedimentation, the solution was diluted 1:1 by the addition of acetone (5 ml) and subsequently evaporation of the solution was performed at 50° C. on a hot plate. The spectrophotometer read-out of the solution, while performing the evaporation procedure changed due to the presence of acetone (FIG. 15). These experiments imply that when there is a trace of acetone it might affect the absorption readout is received.

B. Solubilizing Daunorubicine (Cerubidine Hydrochloride)

Required concentration: 2 mg/ml

Materials and Methods

2 mg Daunorubicine+1 ml Neowater™ was prepared in one vial and 2 mg of Daunorubicine+1 ml RO was prepared in a second vial.

Results

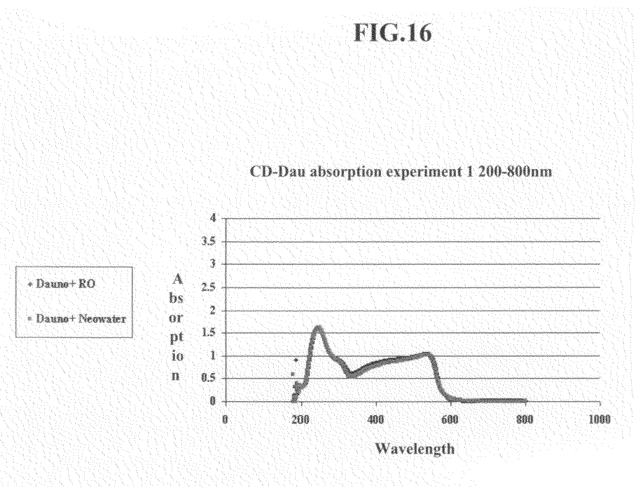

The material dissolved easily both in Neowater™ and RO as illustrated by the spectrophotometer measurements (FIG. 16).

Conclusion

Daunorubicine dissolves without difficulty in Neowater™ and RO.

C. Solubilizing t-boc

Required concentration: 4 mg/ml

Materials and Methods

1.14 ml of EtOH was added to one glass vial containing 18.5 mg of t-boc (an oily material). This was then divided into two vials and 1.74 ml Neowater™ or RO water was added to the vials such that the solution comprised 25% EtOH. Following spectrophotometer measurements, the solvent was evaporated from the solution and Neowater™ was added to both vials to a final volume of 2.31 ml in each vial. The solutions in the two vials were merged to one clean vial and packaged for shipment under vacuum conditions.

Results

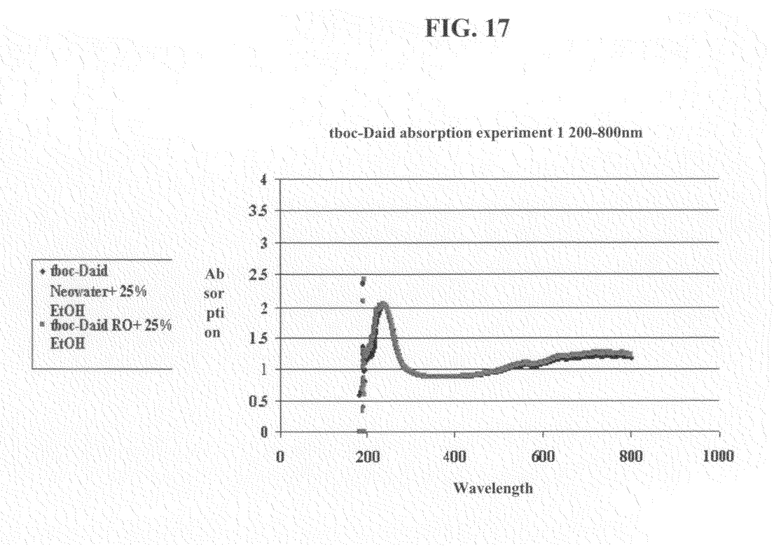

The spectrophotometer measurements are illustrated in FIG. 17. The material dissolved in ethanol. Following addition of Neowater™ and subsequent evaporation of the solvent with heat (50° C.), the material could be dissolved in Neowater™.

Conclusions

The optimal method to dissolve the materials was first to dissolve the material with a solvent (Acetone, Acetic-Acid or Ethanol) followed by the addition of the hydrophilic fluid (Neowater™) and subsequent removal of the solvent by heating the solution and evaporating the solvent.

### Example 10

### Capability of the Liquid Composition Comprising Nanostructures to Dissolve AD-14A and AG-14B

The following experiments were performed in order to ascertain whether the carrier composition comprising nanostructures was capable of dissolving two herbal materials—AG-14A and AG-14B, both of which are known not to dissolve in water at a concentration of 25 mg/ml.

Part 1

Materials and Methods

2.5 mg of each material (AG-14A and AG-14B) was diluted in either Neowater™ alone or a solution comprising 75% Neowater™ and 25% ethanol, such that the final concentration of the powder in each of the four tubes was 2.5 mg/ml. The tubes were vortexed and heated to 50° C. so as to evaporate the ethanol.

Results

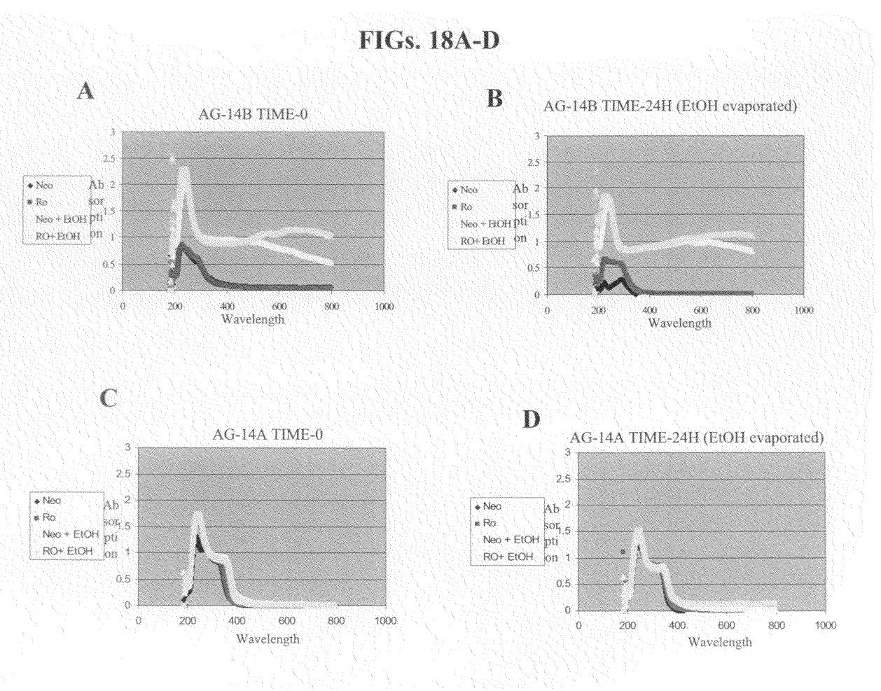

The spectrophotometric measurements of the two herbal materials in Neowater™ in the presence and absence of ethanol are illustrated in FIGS. 18A-D.

Conclusion

Suspension in RO did not dissolve of AG-14B. Suspension of AG-14B in Neowater™ did not aggregate, whereas in RO water, it did.

AG-14A and AG-14B did not dissolve in Neowater/RO.

Part 2

Materials and Methods

5 mg of AG-14A and AG-14B were diluted in 62.5 μl EtOH+187.5 μl Neowater™. A further 62.5 μl of Neowater™ were added. The tubes were vortexed and heated to 50° C. so as to evaporate the ethanol.

Results

Suspension in EtOH prior to addition of Neowater™ and then evaporation thereof dissolved AG-14A and AG-14B.

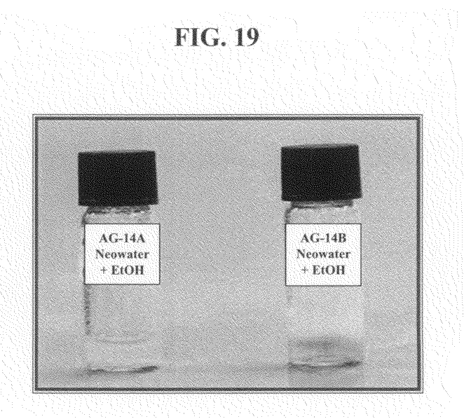

As illustrated in FIG. 19, AG-14A and AG-14B remained stable in suspension for over 48 hours.

### Example 11

### Capability of the Carrier Comprising Nanostructures to Dissolve Peptides

The following experiments were performed in order to ascertain whether the carrier composition comprising nanostructures was capable of dissolving 7 cytotoxic peptides, all of which are known not to dissolve in water. In addition, the effect of the peptides on Skov-3 cells was measured in order to ascertain whether the carrier composition comprising nanostructures influenced the cytotoxic activity of the peptides.

Materials and Methods

Solubilization: All seven peptides (Peptide X, X-5FU, NLS-E, Palm-PFPSYK (CMFU), PFPSYKLRPG-NH₂, NLS-p2-LHRH, and F-LH-RH-palm kGFPSK) were dissolved in Neowater™ at 0.5 mM. Spectrophotometric measurements were taken.

In Vitro Experiment: Skov-3 cells were grown in McCoy's 5A medium, and diluted to a concentration of 1500 cells per well, in a 96 well plate. After 24 hours, 2 μl (0.5 mM, 0.05 mM and 0.005 mM) of the peptide solutions were diluted in 1 ml of McCoy's 5A medium, for final concentrations of 10⁻⁶ M, 10⁻⁷ M and 10⁻⁸ M respectively. 9 repeats were made for each treatment. Each plate contained two peptides in three concentration, and 6 wells of control treatment. 90 μl of McCoy's 5A medium+peptides were added to the cells. After 1 hour, 10 μl of FBS were added (in order to prevent competition). Cells were quantified after 24 and 48 hours in a viability assay based on crystal violet. The dye in this assay stains DNA. Upon solubilization, the amount of dye taken up by the monolayer was quantified in a plate reader.

Results

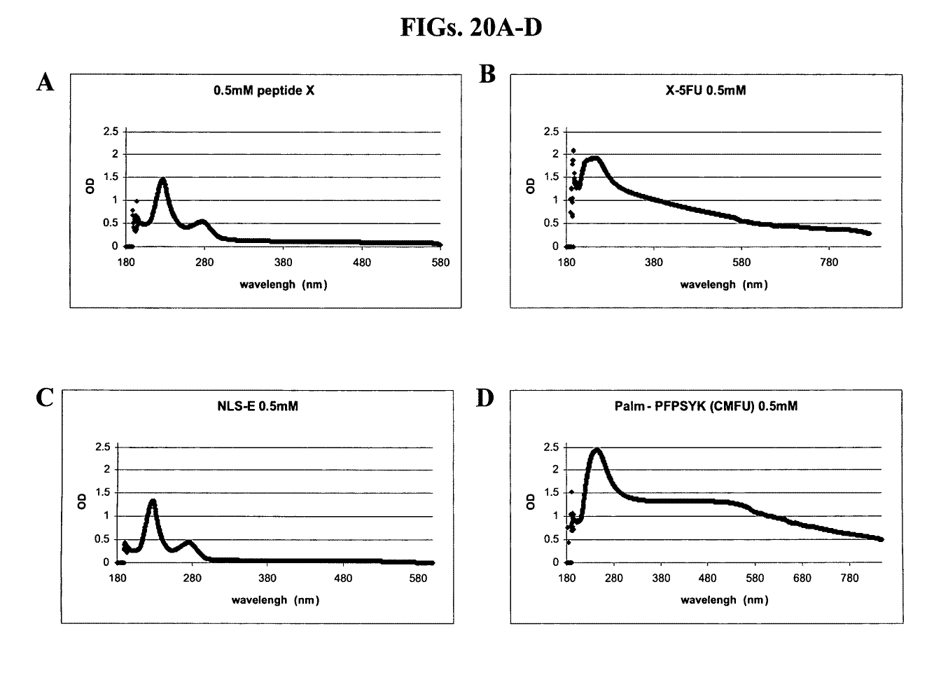

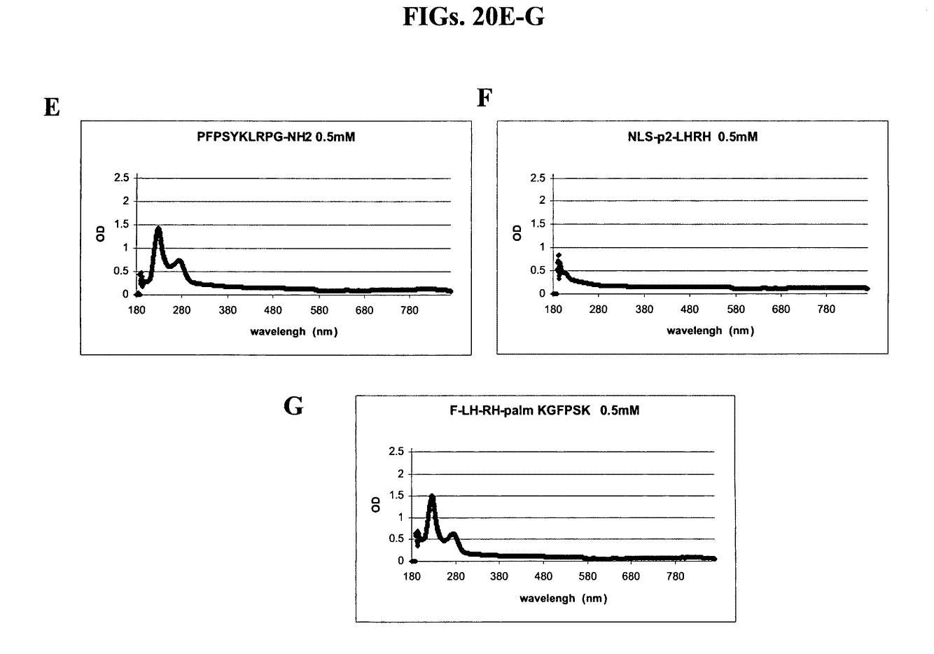

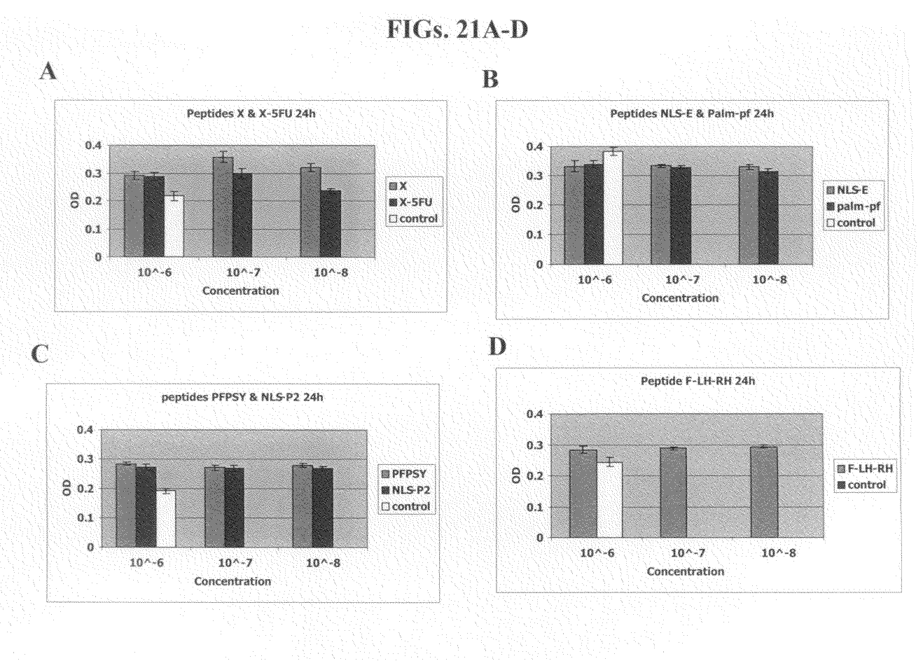

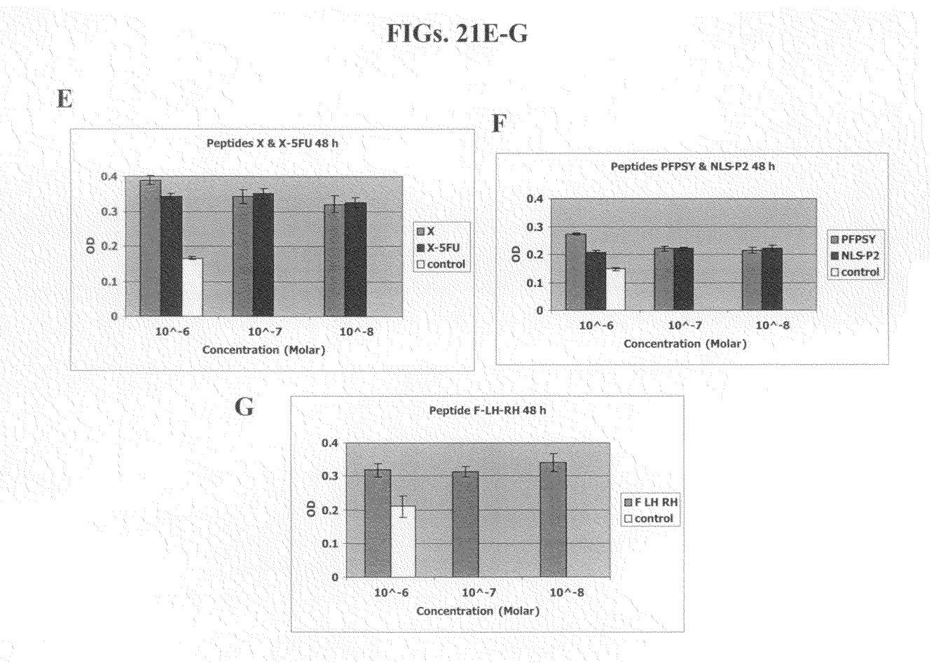

The spectrophotometric measurements of the 7 peptides diluted in Neowater™ are illustrated in FIGS. 20A-G. As illustrated in FIGS. 21A-G, all the dissolved peptides comprised cytotoxic activity.

### Example 12

### Capability of the Liquid Composition Comprising Nanostructures to Dissolve Retinol

The following experiments were performed in order to ascertain whether the liquid composition comprising nanostructures was capable of dissolving retinol.

Materials and Methods

Retinol (vitamin A) was purchased from Sigma (Fluka, 99% HPLC). Retinol was solubilized in Neowater™ under the following conditions.

1% retinol (0.01 gr in 1 ml) in EtOH and Neowater™

0.5% retinol (0.005 gr in 1 ml) in EtOH and Neowater™

0.5% retinol (0.125 gr in 25 ml) in EtOH and Neowater™.

0.25% retinol (0.0625 gr in 25 ml) in EtOH and Neowater™. Final EtOH concentration: 1.5%

Absorbance spectrum of retinol in EtOM: Retinol solutions were made in absolute EtOH, with different retinol concentrations, in order to create a calibration graph; absorbance spectrum was detected in a spectrophotometer.

2 solutions with 0.25% and 0.5% retinol in Neowater with unknown concentration of EtOH were detected in a spectrophotometer. Actual concentration of retinol is also unknown since some oil drops are not dissolved in the water.

Filtration: 2 solutions of 0.25% retinol in Neowater™ were prepared, with a final EtOH concentration of 1.5%.The solutions were filtrated in 0.44 and 0.2 μl filter.

Results

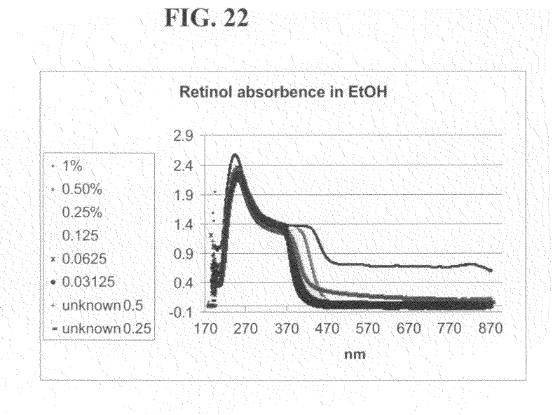

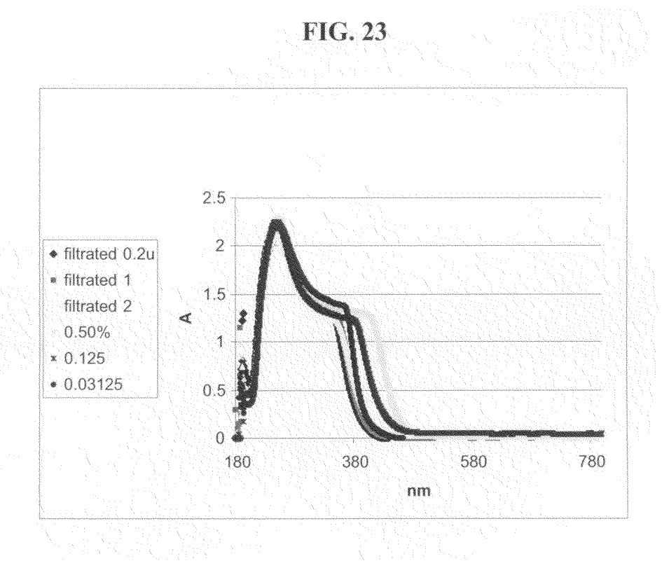

Retinol solubilized easily in alkali Neowater™ rather than acidic Neowater™. The color of the solution was yellow, which faded over time. In the absorbance experiments, 0.5% retinol showed a similar pattern to 0.125% retinol, and 0.25% retinol shows a similar pattern to 0.03125% retinol—see FIG. 22. Since Retinol is unstable in heat; (its melting point is 63° C.), it cannot be autoclaved. Filtration was possible when retinol was fully dissolved (in EtOH). As illustrated in FIG. 23, there is less than 0.03125% retinol in the solutions following filtration. Both filters gave similar results.

### Example 13

### Capability of the Liquid Composition Comprising Nanostructures to Dissolve Material X

The following experiments were performed in order to ascertain whether the liquid composition comprising nanostructures was capable of dissolving material X at a final concentration of 40 mg/ml.

Part 1—Solubility in Water and DMSO

Materials and Methods

In a first test tube, 25 μl of Neowater™ was added to 1 mg of material “X”. In a second test tube 25 μl of DMSO was added to 1 mg of material “X”. Both test tubes were vortexed and heated to 60° C. and shaken for 1 hour on a shaker.

Results

The material did not dissolve at all in Neowater™ (test tube 1). The material dissolved in DMSO and gave a brown-yellow color. The solutions remained for 24-48 hours and their stability was analyzed over time (FIG. 24A-B).

Conclusions

Neowater™ did not dissolve material “X” and the material sedimented, whereas DMSO almost completely dissolved material “X”.

Part 2—Reduction of DMSO and Examination of the Material Stability/Kinetics in Different Solvents Over the Course of Time.

Materials and Methods

6 different test tubes were analyzed each containing a total reaction volume of 25 μl:

1. 1 mg “X”+25 μl Neowater™ (100%).

2. 1 mg “X”+12.5 μl DMSO [symbol] 12.5 μl Neowater™ (50%).

3. 1 mg “X”+12.5 μl DMSO+12.5 μl Neowater™ (50%).

4. 1 mg “X”+6.25 μl DMSO+18.75 μl Neowater™ (25%).

5. 1 mg “X”+25 μl Neowater™+sucrose* (10%).

6. 1 mg+12.5 μl DMSO+12.5 μl dehydrated Neowater™ ** (50%).
* 0.1 g sucrose+1 ml (Neowater™)=10% Neowater™+sucrose

** Dehydrated Neowater™ was achieved by dehydration of Neowater™ for 90 min at 60° C.

All test tubes were vortexed, heated to 60° C. and shaken for 1 hour.

Results

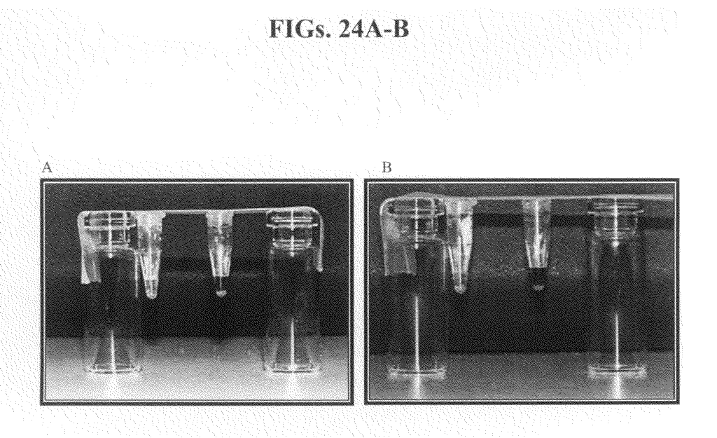

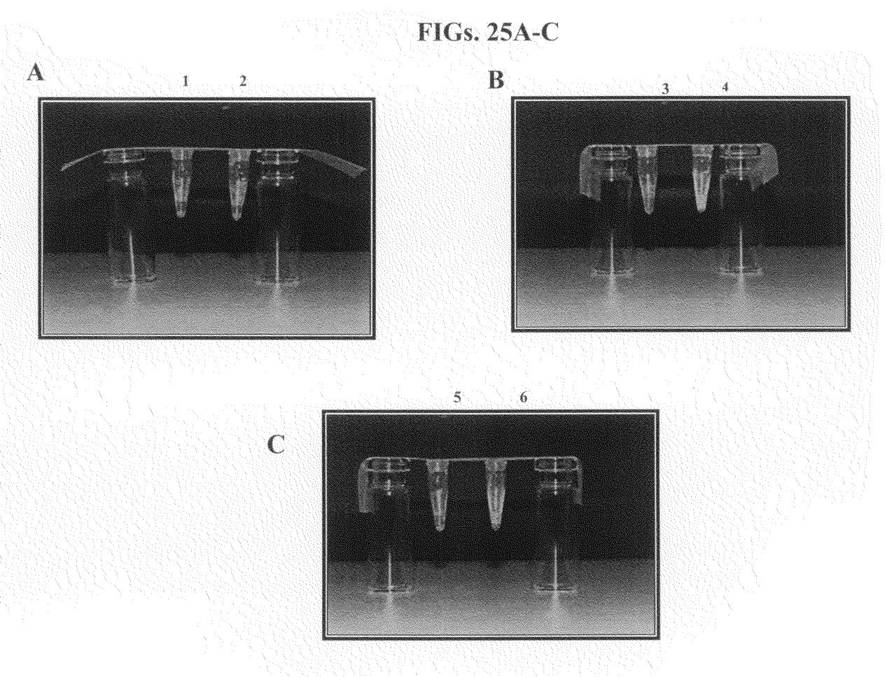

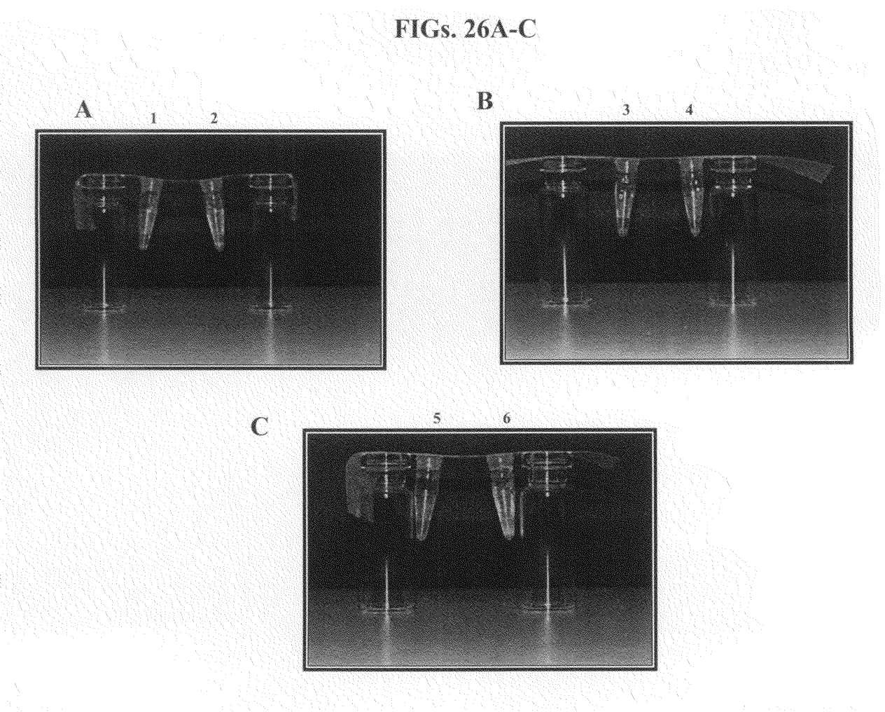

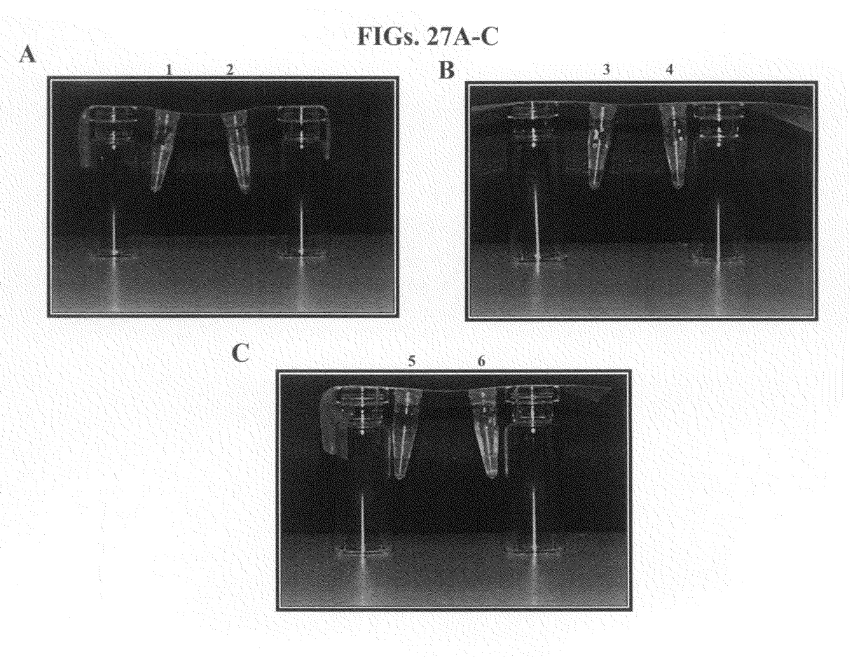

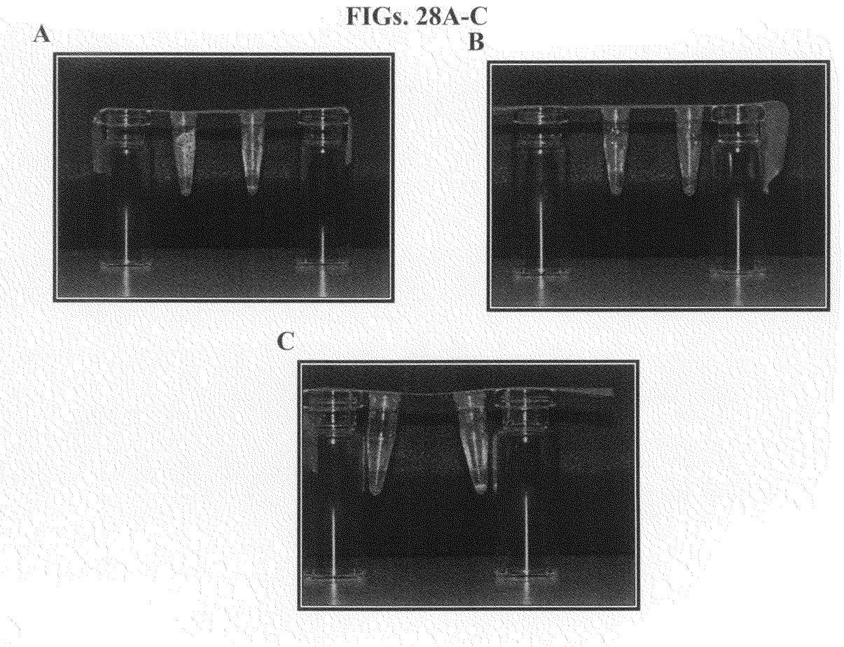

The test tubes comprising the 6 solvents and substance X at time 0 are illustrated in FIGS. 25A-C. The test tubes comprising the 6 solvents and substance X at 60 minutes following solubilization are illustrated in FIGS. 26A-C. The test tubes comprising the 6 solvents and substance X at 120 minutes following solubilization are illustrated in FIGS. 27A-C. The test tubes comprising the 6 solvents and substance X 24 hours following solubilization are illustrated in FIGS. 28A-C.

Conclusion

Material “X” did not remain stable throughout the course of time, since in all the test tubes the material sedimented after 24 hours.

There is a different between the solvent of test tube 2 and test tube 6 even though it contains the same percent of solvents. This is because test tube 6 contains dehydrated Neowater™ which is more hydrophobic than non-dehydrated Neowater™.

Part 3 Further Reduction of DMSO and Examination of the Material Stability/Kinetics in Different Solvents Over the Course of Time.

Materials and Methods

1 mg of material “X”+50 μl DMSO were placed in a glass tube. 50 μl of Neowater™ were titred (every few seconds 5 μl) into the tube, and then 500μl of a solution of Neowater™ (9% DMSO+91% Neowater™) was added.

In a second glass tube, lmg of material “X”+50 μl DMSO were added. 50 μl of RO were titred (every few seconds 5 μl) into the tube, and then 500 μl of a solution of RO (9% DMSO+91% RO) was added.

Results

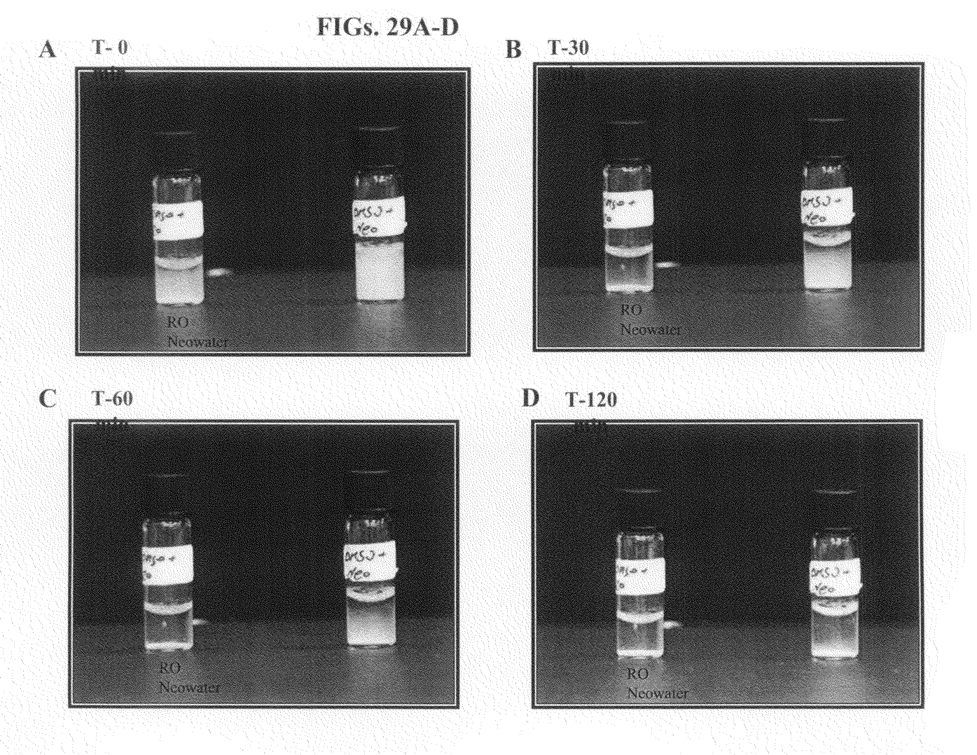

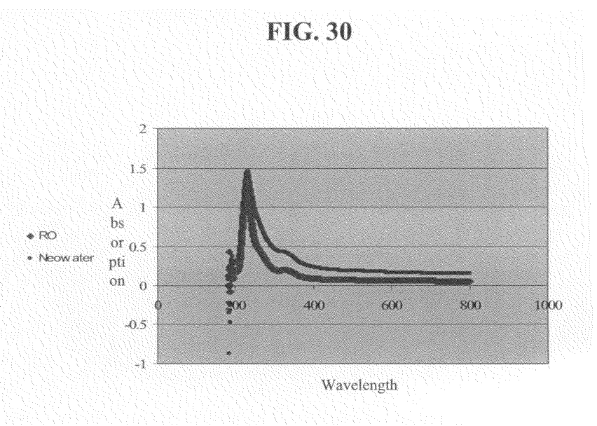

As illustrated in FIGS. 29A-D, material “X” remained dispersed in the solution comprising Neowater™, but sedimented to the bottom of the tube, in the solution comprising RO water. FIG. 30 illustrates the absorption characteristics of the material dispersed in RO/Neowater™ and acetone 6 hours following vortexing.

Conclusion

It is clear that material “X” dissolves differently in RO compare to Neowater™, and it is more stable in Neowater™ compare to RO. From the spectrophotometer measurements (FIG. 30), it is apparent that the material “X” dissolved better in Neowater™ even after 5 hours, since, the area under the graph is larger than in RO. It is clear the Neowater™ hydrates material “X”. The amount of DMSO may be decreased by 20-80% and a solution based on Neowater™ may be achieved that hydrates material “X” and disperses it in the Neowater™.

### Example 14

### Capability of the Liquid Composition Comprising Nanostructures to Dissolve SPL 2101 and SPL 5217

The following experiments were performed in order to ascertain whether the liquid composition comprising nanostructures was capable of dissolving material SPL 2101 and SPL 5217 at a final concentration of 30 mg/ml.

Materials and Methods

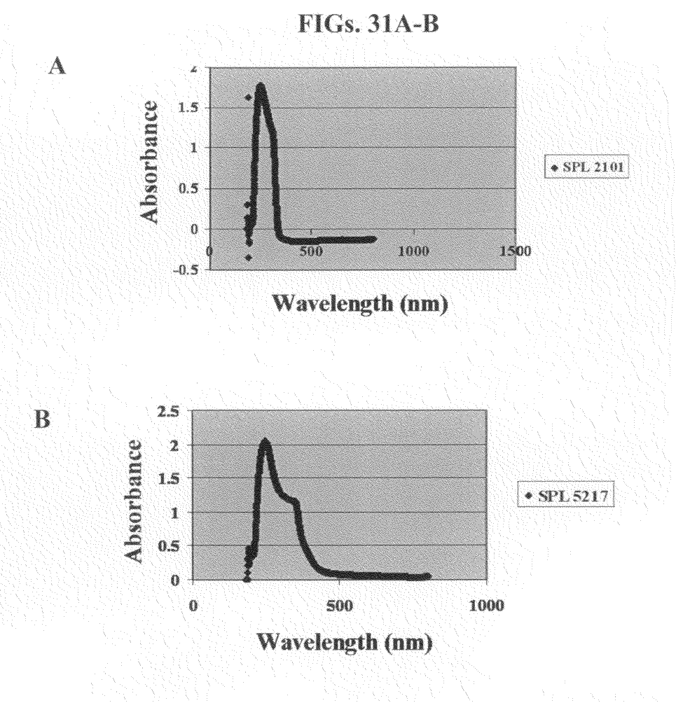

SPL 2101 was dissolved in its optimal solvent (ethanol)—FIG. 31A and SPL 5217 was dissolved in its optimal solvent (acetone)—FIG. 31B. The two compounds were put in glass vials and kept in dark and cool environment. Evaporation of the solvent was performed in a dessicator and over a long period of time Neowater™ was added to the solution until there was no trace of the solvents.

Results

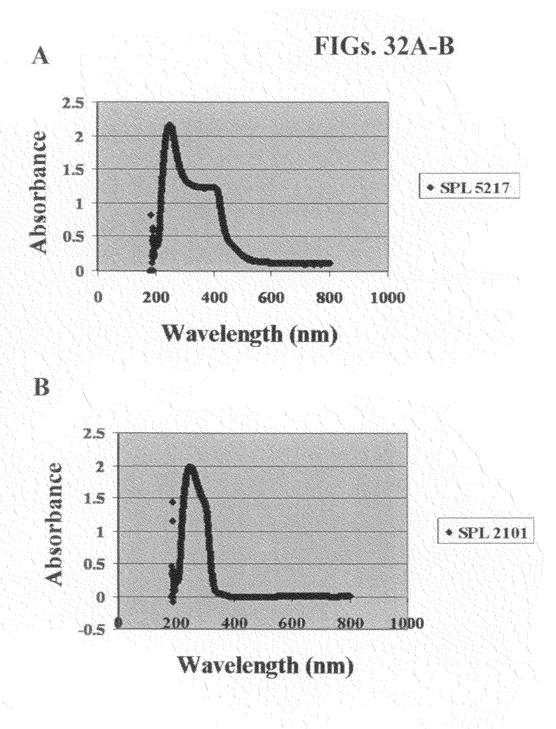

SPL 2101 & SPL 5217 dissolved in Neowater™ as illustrated by the spectrophotometer data in FIGS. 32A-B.

### Example 15

### Capability of the Liquid Composition Comprising Nanostructures to Dissolve Taxol

The following experiments were performed in order to ascertain whether the carrier composition comprising nanostructures was capable of dissolving material taxol (Paclitaxel) at a final concentration of 0.5 mM.

Materials and Methods

Solubilization: 0.5 mM Taxol solution was prepared (0.0017 gr in 4 ml) in either DMSO or Neowater™ with 17% EtOH. Absorbance was detected with a spectrophotometer.

Cell viability assay: 150,000 293T cells were seeded in a 6 well plate with 3 ml of DMEM medium. Each treatment was grown in DMEM medium based on RO or Neowater™. Taxol (dissolved in Neowater™ or DMSO) was added to final concentration of 1.666 μM (10 μl of 0.5 mM Taxol in 3 ml medium). The cells were harvested following a 24 hour treatment with taxol and counted using trypan blue solution to detect dead cells.

Results

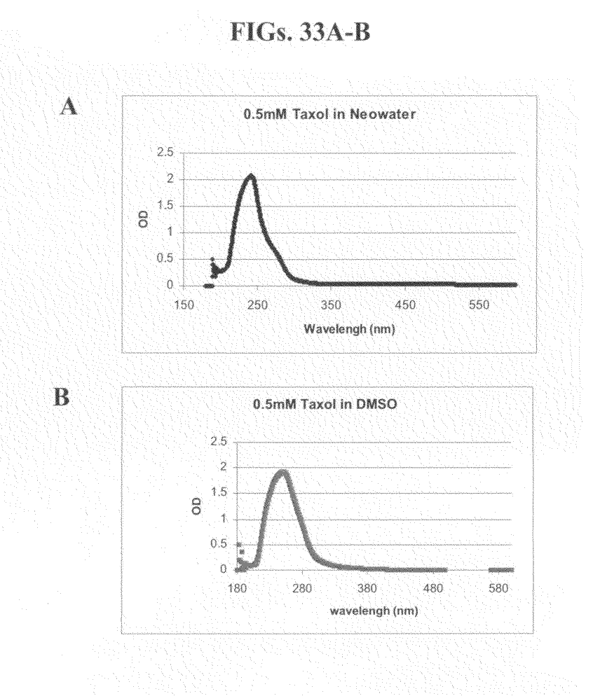

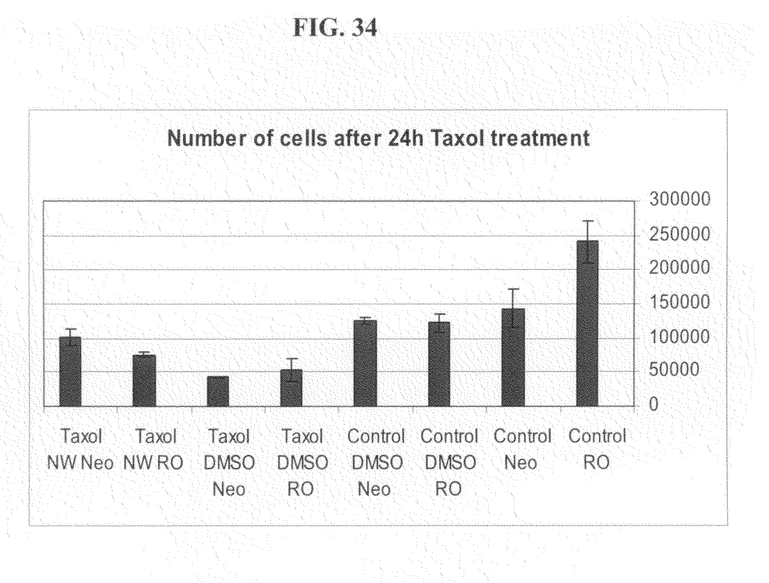

Taxol dissolved both in DMSO and Neowater™ as illustrated in FIGS. 33A-B. The viability of the 293T cells following various solutions of taxol is illustrated in FIG. 34.

Conclusion

Taxol comprised a cytotoxic effect following solution in Neowater™.

### Example 16

### Stabilizing Effect of the Liquid Composition Comprising Nanostructures

The following experiment was performed to ascertain if the liquid composition comprising nanostructures effected the stability of a protein.

Materials and Methods

Two commercial Taq polymerase enzymes (Peq-lab and Bio-lab) were checked in a PCR reaction to determine their activities in ddH₂O (RO) and carrier comprising nanostructures (Neowater™—Do-Coop technologies, Israel). The enzyme was heated to 95° C. for different periods of time, from one hour to 2.5 hours.

2 types of reactions were made:

Water only—only the enzyme and water were boiled.

All inside—all the reaction components were boiled: enzyme, water, buffer, dNTPs, genomic DNA and primers.

Following boiling, any additional reaction component that was required was added to PCR tubes and an ordinary PCR program was set with 30 cycles.

Results

As illustrated in FIGS. 35A-B, the carrier composition comprising nanostructures protected the enzyme from heating, both under conditions where all the components were subjected to heat stress and where only the enzyme was subjected to heat stress. In contrast, RO water only protected the enzyme from heating under conditions where all the components were subjected to heat stress.

### Example 17

### Further Illustration of the Stabilizing Effect of the Carrier Comprising Nanostructures

The following experiment was performed to ascertain if the carrier composition comprising nanostructures effected the stability of two commercial Taq polymerase enzymes (Peq-lab and Bio-lab).

Materials and Methods

The PCR reactions were set up as follows:

Peq-lab samples: 20.4 μl of either the carrier composition comprising nanostructures (Neowater™—Do-Coop technologies, Israel) or distilled water (Reverse Osmosis=RO).

0.1 μl Taq polymerase (Peq-lab, Taq DNA polymerase, 5 U/μl)

Three samples were set up and placed in a PCR machine at a constant temperature of 95° C. Incubation time was: 60, 75 and 90 minutes.

Following boiling of the Taq enzyme the following components were added:
2.5 μl 10× reaction buffer Y (Peq-lab)
0.5 μl dNTPs 10 mM (Bio-lab)
1 μl primer GAPDH mix 10 pmol/μl
0.5 μl genomic DNA 35 μg/μl

Biolab Samples

18.9 μl of either carrier comprising nanostructures (Neowater™—Do-Coop technologies, Israel) or distilled water (Reverse Osmosis=RO).

0.1 μl Taq polymerase (Bio-lab, Taq polymerase, 5 U/μl)

Five samples were set up and placed in a PCR machine at a constant temperature of 95° C. Incubation time was: 60, 75, 90 120 and 150 minutes.

Following boiling of the Taq enzyme the following components were added:
2.5 μl TAQ 10× buffer Mg-free (Bio-lab)
1.5 μl MgCl₂ 25 mM (Bio-lab)
0.5 μl dNTPs 10 mM (Bio-lab)
1 μl primer GAPDH mix (10 pmol/μl)
0.5 μl genomic DNA (35 μg/μl)

For each treatment (Neowater or RO) a positive and negative control were made. Positive control was without boiling the enzyme. Negative control was without boiling the enzyme and without DNA in the reaction. A PCR mix was made for the boiled taq assays as well for the control reactions.

Samples were placed in a PCR machine, and run as follows:

PCR Program:

1. 94° C. 2 minutes denaturation

2. 94° C. 30 seconds denaturation

3. 60° C. 30 seconds annealing

4. 72° C. 30 seconds elongation

repeat steps 2-4 for 30 times

5. 72° C. 10 minutes elongation

Results

As illustrated in FIG. 36, the liquid composition comprising nanostructures protected both the enzymes from heat stress for up to 1.5 hours.

### Example 18

### Further Evidence that the Liquid Composition Comprising Nanostructures is Capable of Dissolving Taxol

The following experiments were performed in order to ascertain whether the carrier composition comprising nanostructures was capable of dissolving material taxol (Paclitaxel) at a final concentration of 0.5 mM in the presence of 0.08% ethanol.

Materials and Methods

Solubilization: 0.5 mM Taxol solution was prepared (0.0017 gr in 4 ml). Taxol was dissolved in ethanol and exchanged to Neowater™ using an RT slow solvent exchange procedure which extended for 20 days. At the end of the procedure, less than 40% ethanol remained in the solution, leading to 0.08% of ethanol in the fmal administered concentration. The solution was sterilized using a 0.2 μm filter. Taxol was separately prepared in DMSO (0.5 mM). Both solutions were kept at −20° C. Absorbance was detected with a spectrophotometer.

Cell viability assay: 2000 PC3 cells were seeded per well of a 96-well plate with 100 μl of RPMI based medium with 10% FCS. 24 hours post seeding, 2 μl, 1 μl and 0.5 μl of 0.5 mM taxol were diluted in 1 ml of RPMI medium, reaching a final concentration of 1 μM, 0.5 μM and 0.25 μM respectively. A minimum number of eight replicates were run per treatment. Cell proliferation was assessed by quantifying the cell density using a crystal violet colorimetric assay 24 hours after the addition of taxol.

24 hours post treatment, the cells were washed with PBS and fixed with 4% paraformaldehyde. Crystal violet was added and incubated at room temperature for 10 minutes. After washing the cells three times, a solution with 100 M Sodium Citrate in 50% ethanol was used to elute the color from the cells. Changes in optical density were read at 570 nm using a spectrophotometric plate reader. Cell viability was expressed as a percentage of the control optical density, deemed as 100%, after subtraction of the blank.

Results

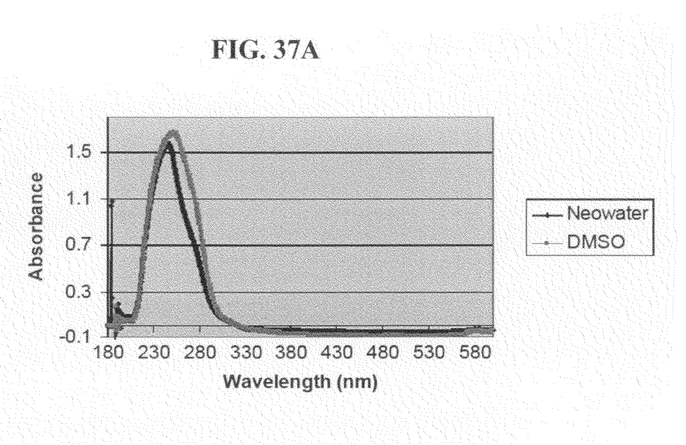

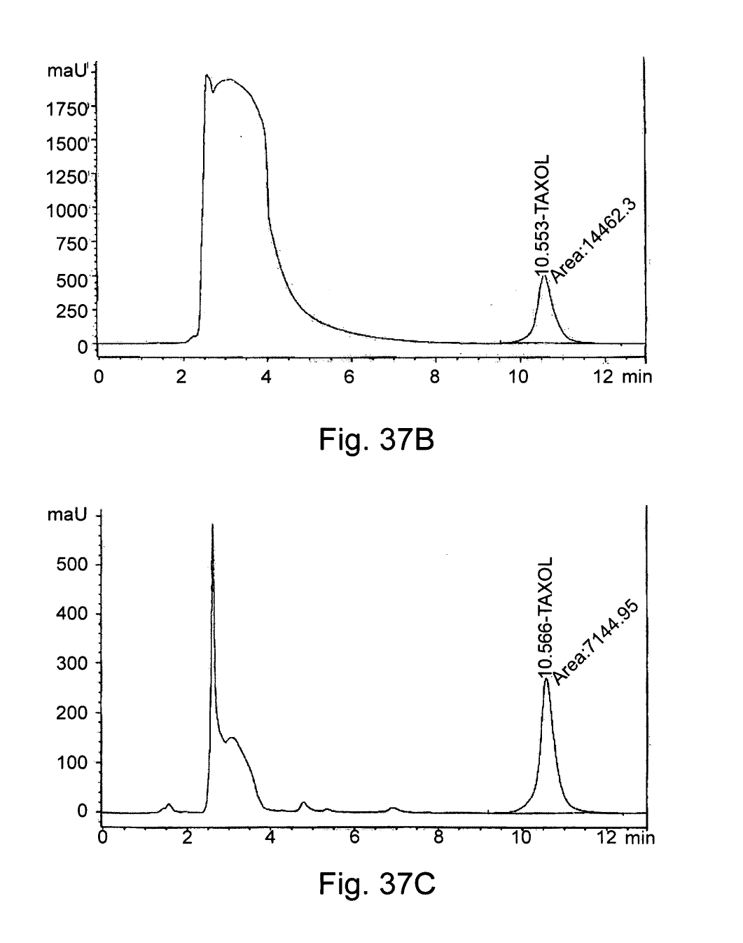

The spectrophotmetric absorbance of 0.5 mM taxol dissolved in DMSO or Neowater™ is illustrated in FIG. 37A. FIGS. 37B-C are HPLC readouts for both formulations. Measurements showed no structural changes in the formulation of taxol dispersed in Neowater™ following a 6 month storage period.

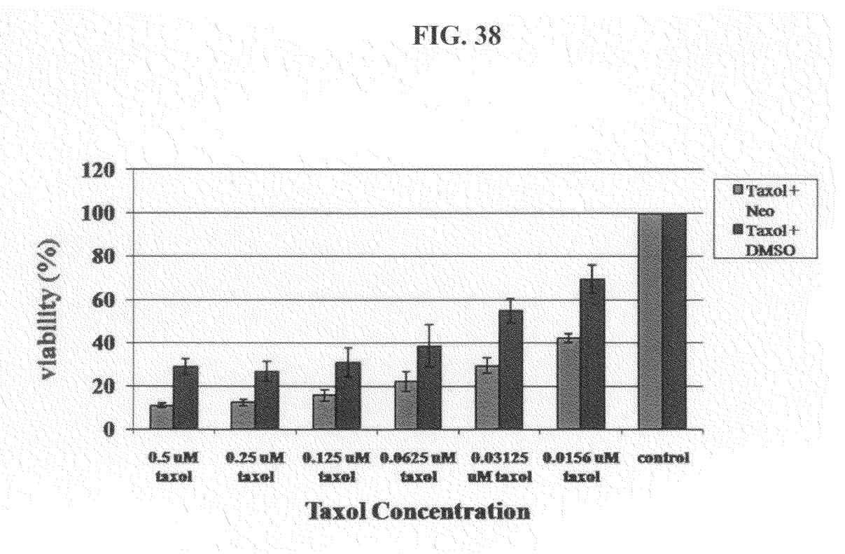

The results of taxol-induced loss of cell viability is illustrated in FIG. 38 following dissolving in DMSO or Neowater™.

Conclusion

Taxol dissolved in Neowater™ (with 0.08% ethanol in the final working concentration) showed similar in vitro cell viability/cytotoxicity on a human prostate cancer cell line as taxol dissolved in DMSO.

### Example 19

### Further Experiment Illustrating the Effect of Water Comprising Nanostructures on the Isolation of Human Hybridomas

The following experiments were performed in order to ascertain whether water comprising nanostructures affects the first stage of monoclonal antibody production—isolation of hybridomas.

Reagents for cell growth: All the media and supplements for cell growth were purchased from GIBCO BRL, Life Technologies. RPMI 1640 and DMEM were purchased in powder form and reconstituted either in NPD or in DI water. After reconstitution sodium bicarbonate was added to the media according to the manufacturers' recommendation, and there was no further adjustment of pH. Prior to use, all the media were filter-sterilized through a 0.22 μm filter (Millipore). For the growth of hybridoma cells, RPMI was supplemented with 10% fetal calf serum, L-glutamine (4 mM), penicillin (100 U/mL), streptomycin (0.1 mg/mL), MEM-vitamins (0.1 mM), non-essential amino acids (0.1 mM) and sodium pyruvate (1 mM). All the supplements mentioned above were bought in a liquid form and used as is from the manufacturer (meaning, they were diluted into Neowater™ or control water (DI based media—18.2 mega ohm ultrapure deionized water (DI water, UHQ PS, ELGA Labwater). 8-Azaguanine, HT and HAT were purchased from Sigma and reconstituted from powder form with NPD or DI RPMI. DMEM used for human primary fibroblasts and CHO cells growth was supplemented with 10% fetal calf serum, L-glutamine (4 mM), penicillin (100 U/mL), streptomycin (0.1 mg/mL). Hybridoma cloning factor was bought from BioVeris.

Chemical reagents: Powdered PBS was obtained from GIBCO BRL, Life Technologies. PEG-1450 (P5402, Sigma) was purchased from Sigma and reconstituted with sterile PBS based on Neowater™ or on control water (50% w/v). The preparation was adjusted to pH 7.2, DMSO (v/v)(Sigma) was added to 10% followed by sterile filtration of the PEG solution through a 0.45 μm filter (Millipore). Hanks balanced salt solution was bought from Biological Industries Beit-HaEmek LTD, Israel and used as is for Neowater™ and control-based experiments. Carbonate-bicarbonate buffer (0.05 M, pH=9.6) for ELISA plate-coating, OPD (used in 0.4 mg/mL) and phosphate-citrate buffer (0.05 M, pH=5.0) were bought from Sigma.

Antibodies: Goat anti-human IgM/IgG and HRP-conjugated goat anti-human IgM/IgG were purchased from Jackson ImmunoResearch. Standard human IgM/IgG were bought from Sigma.

Cells: All cells used in these experiments (MFP-2, CHO and primary human fibroblasts) were maintained for a week in either Neowater™ and control-based media so that the cells were adapted to the media prior to experimentation. In addition, the fusion partner cell line MFP-2 was maintained in RPMI 1640 with the addition of fetal bovine serum and additives along with 8-azaguanine to maintain the HGPRT minus phenotype. Primary human fibroblasts were obtained from the ATCC and maintained in DMEM. The CHO cell line was maintained in DMEM. All cell culture was performed in complete media, which consists of culture media with the addition of fetal calf serum, glutamine and penicillin/streptomycin. For the MFP-2 cell line vitamins, nonessential amino acids and pyruvate were also added in complete medium.

Methods

Cell Fusion: The chemical fusion technique [Kohler G, Milstein C (1975) Nature 256: 495-497] with PEG 1450 was employed, which acts as a fusogen, for creation of hybridomas with human peripheral blood lymphocytes. PEG 1450 is typically prepared in PBS with the addition of 10% DMSO. For these experiments, Neowater™ was used to prepare PBS, which was then used to make a PEG/DMSO solution; as a control preparation PEG prepared in control water based PBS was used. For all fusion experiments comparing Neowater™ to control water, all reagents were prepared in either Neowater™ or control water except for fetal bovine serum and concentrates of supplements. In addition, dilution of cells in Hanks balanced salts (HBSS)(see below), following fusion with PEG-1450, was performed with a purchased liquid form of HBSS (Beit HaEmek, Israel) and used as is from the manufacturer.

For production of hybridomas, human peripheral blood mononuclear cells (PBMC) were isolated from 40 mL of freshly drawn whole blood, purified with Histopaque 1077 (Sigma), and washed 4 times in control water based culture medium without serum. The MFP-2 fusion partner cells were either grown in Neowater™ or control water based media and then washed with the respective media 4 times without serum. For each experiment a single batch of PBMC was divided into two equal fractions, one of which was used for Neowater™ and the other for control water fusions. Next, MFP-2 and PBMC were mixed in either Neowater™ or control water based media without serum and pelleted. PEG-1450 pre-warmed to 37° C. was then added at 300 μL for 10-200×10⁶ of mixed cells. The cell mixture was incubated with PEG for 3 minutes with constant shaking. PEG was then diluted out of the cell mixture with Hanks balanced salt solution and complete RPMI (prepared in either Neowater™ or control water). To the resultant cell suspension were added: fetal calf serum (10%) and HT (×2). The hybridomas that were generated in this process were cultured in 96-well plates (cell density—2×10⁶ lymphocytes/well) in complete RPMI with HAT selection. The screening of the supernatants for immunoglobulin production was performed after the hybridoma cells occupied approximately ¼ of the well.

Sandwich ELISA: A sandwich ELISA was used to screen hybridoma supernatants for IgM/IgG. Briefly, a capturing antibody (goat anti-human IgM/IgG) was prepared in a carbonate/bicarbonate buffer and applied on a 96-well plate in a concentration of 100 ng/100 μL/well. The plate was then incubated overnight at 4° C. All the following steps were performed at room temperature. After 1 hour of blocking with 0.3% dry milk in PBS, the supernatants from the hybridomas were applied for 1.5 hours. Human serum diluted 1:500 in PBS was used as a positive control. For a background and as a negative control hybridoma growth medium was used. The secondary antibody (HRP-conjugated goat anti-human IgM/IgG) was prepared in blocking solution at a concentration of 1:5000 and incubated for 1 hour. To produce a calorimetric reaction the plates were incubated with OPD in phosphate-citrate buffer, containing 0.03% H₂O₂. The color reaction was stopped with 10% HCl after 15 minutes. The reading and the recording of the reaction were performed with a Multiscan-Ascent (Thermo Scientific) ELISA reader using the 492 nm wavelength filter. All reagents used were standard with the exception of the sandwich layer, which consisted of the NPD or DI based hybridoma supernatant.

Cloning: Two hundred cells of a chosen clone were diluted in a volume of 10 mL of media and seeded in a 96-well plate (100 μL/well), so that on average the wells contained 1-2 cells. The cells were incubated and periodically fed and microscopically monitored for clonal growth. When a clone occupied ¼-½ of the well, its supernatant was analyzed. The efficiency of cloning was expressed in a number of viable clones per plate. Ten percent HCF (hybridoma cloning factor) was added according to the experimental design.

Cell growth assay: Growth of primary and immortalized cell lines was monitored with a crystal violet dye retention assay. A fixed number of cells were seeded in 96-well plates in multiple repeats. Cell growth was stopped by fixation in 4% formaldehyde. Fixed cells were then stained with 0.5% crystal violet followed by extensive washing with water. The retained dye was extracted in 100 μL/well of 0.1 M sodium citrate in 50% ethanol (v/v). The absorbance of the wells was then read at 550 nm with a Multiscan-Ascent microplate reader and the appropriate filter.

Primary human fibroblast culture: Starting at passage twenty, human fibroblasts were cultured and passed every week as long as the cells displayed typical fibroblast morphology and their number did not drop below the initially seeded amount. The number of passages and calculated population doublings were recorded. The morphology and viability of the cells were monitored microscopically. Human fibroblasts used in these experiments were generally at a population doubling of 25.

Data analysis: The statistical significance of difference in the efficiency of fusion and cloning between Neowater™ or control-based experiments was determined by the Chi-square test. The results of the growth test with primary human fibroblasts were analyzed by an unpaired Students' t-test. Statistical p-values<0.05 were considered significant.

Results

Neowater™ Enhances Efficiency of Hybridoma Formation for Production of Human Monoclonal Antibodies

Results of chemical fusion experiments are presented in FIG. 39. For these experiments PBMCs from a single individual were divided into two groups after purification for fusion in either a Neowater™ or control based environment. A statistically significant difference in the yield of hybridomas between NPD and DI environments was witnessed. There was a clear tendency for a greater yield of hybridomas in the Neowater™ based fusion experiments as compared to the parallel fusions in control based media. The percent of enhancement was calculated by the formula [(number of hybridomas in Neowater™ fusion/number of hybridomas in control water fusion)×100%-100%] and these results are depicted in FIG. 39. The extent of enhancement is variable, and within a series of eight fusion experiments varied from 22 to 227 percent. Although the increased efficiency of fusion in NPD is variable, this is not unexpected as each fusion was performed with lymphocytes from a different donor. As such, magnitude of the effect of a NPD aqueous environment on hybridoma formation is a function to some extent of the genetic background.

Increased Yield of Hybridoma Subclones in NPD Water

One of the crucial steps in the process of monoclonal antibody production is the isolation of a stable subclone from a primary hybridoma population found to be positive for secretion of a specific monoclonal antibody. This is typically achieved by serially subcloning of a specific primary hybridoma clone. The purpose of subcloning, which involves seeding 1-2 cells per well, is to produce clones of a single origin, which are genetically stable and produce a unique monoclonal antibody. During this process, hybridoma cells can die due to genetic instability or proliferate but lose their capacity to produce antibodies. To overcome these difficulties hybridoma cloning factor (HCF) is used, which consists of macrophage conditioned media containing a variety of factors that facilitate clone outgrowth and stabilization. However, since the fusion partner cell line used is of myeloma origin, the hybridomas that are produced with it likely secrete autocrine factors that promote their own clonal expansion. The autocrine action of these factors, however, is not apparent in standard in vitro culture due to their relatively low concentration. The ability of Neowater™ based media to enhance the bioavailability was tested, and hence autocrine activity, of these secreted factors, through increase in the cell-localized concentration. This was best achieved through subcloning primary hybridoma cells in control water versus Neowater™ based media and also observing the effect of adding HCF to both cloning medias.

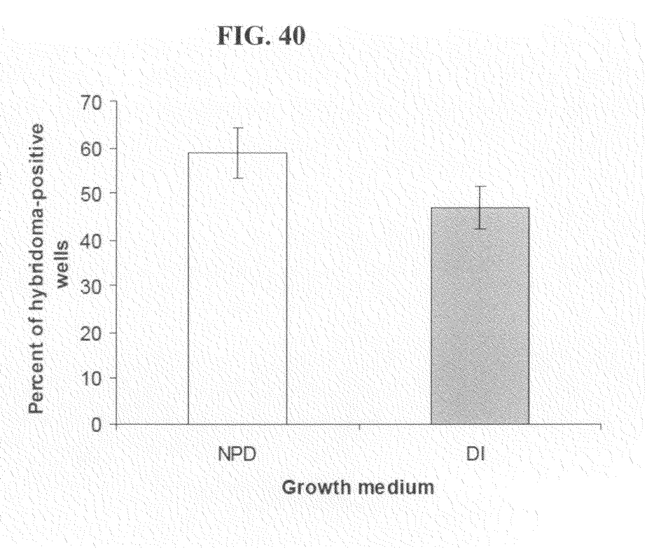

Following fusion of PBMC with MFP-2 and outgrowth of primary hybridoma clones, antibody-producing hybridomas were identified and subcloned in either Neowater™ or control water based media with supplements. The results of these experiments are displayed in Table 6. Overall, for each primary hybridoma tested, a greater clonal outgrowth in Neowater™ based media was observed as compared to control water based media. When HCF was added to both Neowater™ and control water based media, a similar percentage increase in the number of clones in both formulations was noted. Finally, as shown in FIG. 40, clonability of cells from semi-stable clones is also enhanced in NPD based media.

TABLE 6

| | Treatment | | | |
|---|---|---|---|---|
| | DI-RPMI + | NPD-RPMI + | DI- | NPD- |
| | HCF | HCF | RPMI | RPMI |
| Num. of hybridoma- | 28 (29) | 46 (48) | 13 (13) | 25 (26) |
| positive wells out of | | | | |
| 96 wells (%) | | | | |

Table 6: From a single primary antibody-producing hybridoma clone, 200 cells were counted, added to a volume of 10 mL and seeded over a 96-well plate (on average 1-2 cells/100 μL/well). The table presents numbers (and percents in parenthesis) of viable subclones, which were counted microscopically in each treatment.

### Chi-Square Analysis:

DI-RPMI+HCF versus DI-RPMI p=0.008; DI-RPMI+HCF versus NPD-RPMI p=not significant; DI-RPMI+HCF versus NPD-RPMI+HCF p=0.008; NPD-RPMI+HCF versus DI-RPMI p<0.00001; NPD-RPMI+HCF versus NPD-RPMI p=0.002; NPD-RPMI versus DI-RPMI p=0.03.

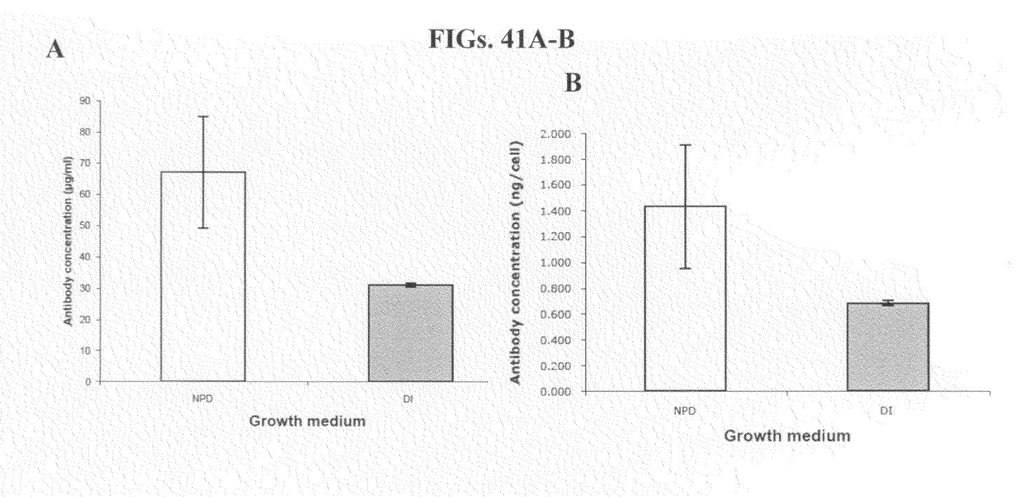

Increased secretion of monoclonal antibodies from hybridomas grown in NPD water To study the effect of a Neowater™ aqueous environment on secretion of monoclonal antibodies the production of human monoclonal antibodies from several stabilized hybridoma clones was studied. Hybridoma clones that have been stably producing antibodies for over 5 years were grown in control water based medium and then two parallel cultures were prepared from it, one in Neowater™ and the other in control water based medium. Following a period of several days of adaptation, cells were seeded at equivalent densities in replicate and after five days of growth supernatants were harvested and antibody concentrations were measured by standard sandwich ELISA. The results of one of these experiments are presented in FIG. 41A, although all showed similar results. As is evident from the graph, although the yields from the replicate Neowater™ based cultures were somewhat variable (Neowater™ based culture concentrations ranged from 101 to 40 μg/mL, whereas in control water the range was much narrower: 30-32 μg/mL), there was overall a greater yield of monoclonal antibody in the Neowater™ based media. However, some cells grow faster in Neowater™ based media (see below). Thus this result might not reflect greater secretion per cell but rather greater proliferation of cells with similar secretion. To obviate this bias the antibody concentration was normalized to the number of cells in each culture (FIG. 41B). Following normalization the results are similar to the batch concentrations, and indicate that the secretion of monoclonal antibody in Neowater™ based media is roughly twice that obtained in control water based media.

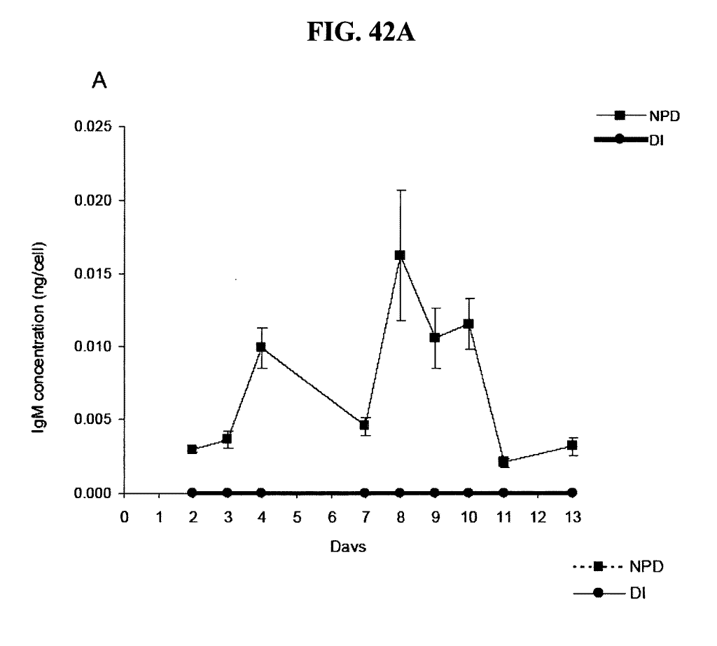

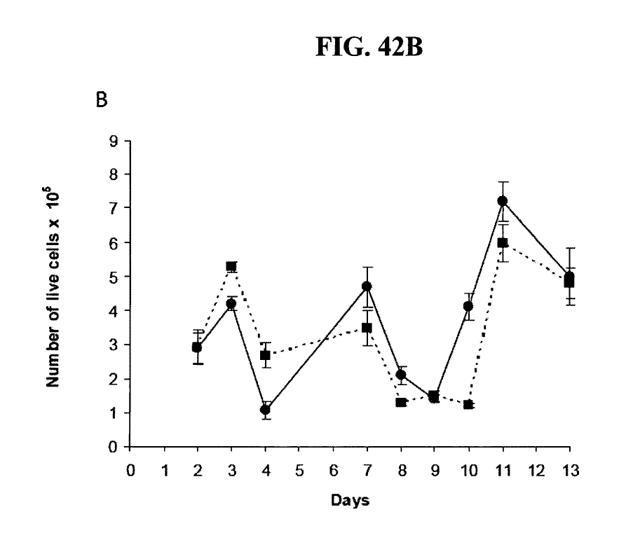

To further study the effect of Neowater™ media on secretion, the secretion of monoclonal antibody was examined in cultures grown in reduced serum. This experiment enabled the examination of secretion in cultures that were less active (relatively quiescent as compared to complete medium with 10% fetal bovine serum) but still metabolically active, thereby eliminating some of the proliferative bias of the Neowater™ based media. FIG. 42 presents the results of these experiments, where both daily antibody concentrations and viable cell counts of a stable hybridoma clone grown in 3% FCS, in replicate, were quantitated. In Neowater™ culture the antibody concentration changed along with the quantity of viable cells in culture. Cell proliferation and variation in number was also a function of the replacement of medium and feedings (days 4 and 10 medium was added to the culture to feed cells and on day 6 the medium was completely replaced), which also impacted the concentration of antibody. In contrast, cells in the control water culture kept proliferating but failed to produce any measurable quantity of antibody. The graphs in FIG. 42 depict typical relationships between hybridoma cell proliferation and the antibody content of the culture. In general, in Neowater™ based media the quantity of antibody increases following an increase in cell number, which occurs following a proliferative burst after feeding. The pattern of the graphs reflects the dilutional effect on antibody concentration from media replacement and also the concomitant leap in cell proliferation (day 6 after medium replacement).

Cell Proliferation in a Neowater™ Based Aqueous Environment

The results of the previous experiments with the hybridoma clones suggested that Neowater™ based media affected clonal expansion and survivability of human hybridoma cells. To further examine this hypothesis, the growth of the immortal CHO cell line and primary human fibroblasts was studied in Neowater™ and control water based media.

Immortal Cell Lines Grow Faster in Neowater™

CHO cells were grown in Neowater™ and control water based complete DMEM parallel cultures. Cells were seeded at an initial density of 1.5×10⁶ per 10-cm Petri dish in replicate cultures. After overnight growth they were detached by trypsinization and counted. The results are presented in FIGS. 43A-C, which demonstrates that in Neowater™ medium the cells grew faster by an average of nearly 30%. To examine the effect of serum depletion on CHO cell growth, cells were seeded in parallel Neowater™ and control water based cultures in replicate with either 5% or 1% FCS. In these experiments cell mass was quantitated by means of crystal violet dye retention assay. The results of this experiment, illustrated in FIG. 43A-C indicate that under serum reduced conditions cells grow faster in Neowater™ based media as compared to a control water based media.

Primary Human Fibroblasts Grow Slower in NPD Water

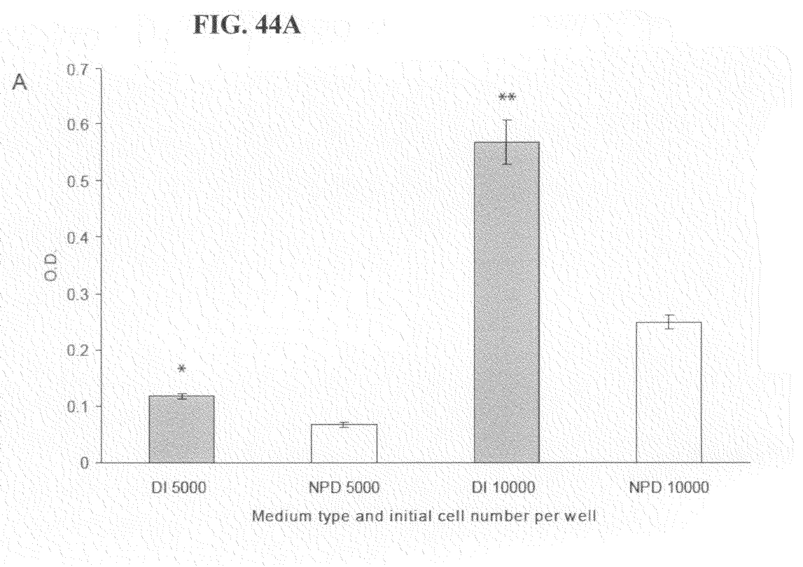

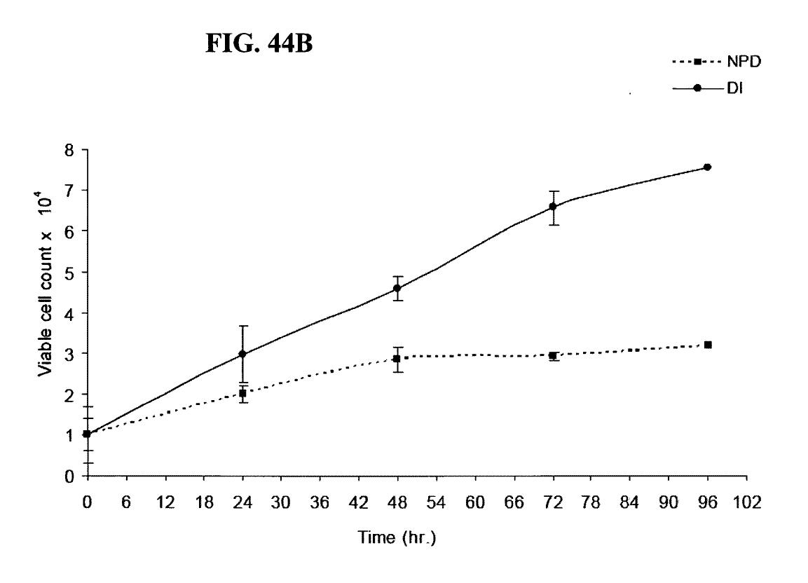

Primary human fibroblasts at a relatively low passage (twenty population doublings) were first cultured in Neowater™ and control water based media to adapt the cells to their respective growth media. Since primary fibroblasts are sensitive to cell density, the effect of Neowater™ versus control water based media was examined on cell proliferation with different initial seeding density. In a 96-well plate, two cell densities were seeded in replicate wells in both Neowater™ and control water based media, five and ten thousand cells per well. After an overnight growth the plates were analyzed with a crystal violet dye retention assay. The results of this assay are depicted in FIG. 44A. At both cell densities, fibroblasts grown in control water based media proliferated faster than in Neowater™ based media. This difference was found to be highly statistically significant (p<<0.0001) The calculation of the percentage of a difference showed that at the higher density the difference between treatments was more pronounced (56%) than at the lower density (44%).

To further study the effect of NPD based media on primary human fibroblast growth, the growth of fibroblasts in control water and Neowater™ based media over eight days in replicate cultures. Cells were seeded at ten thousand cells per well in replicate parallel cultures, since in the previous experiment fibroblasts proliferated well at this density in DI based media. Growth curves from this experiment are displayed in FIG. 44B. As is evident from the curves, primary human fibroblasts proliferated poorly in Neowater™ based media as compared to control water based media. This indicates that the environment in Neowater™ is less favorable for primary fibroblast outgrowth, and suggests that cell-cell sensing is somehow impaired since fibroblast proliferation is a function of cell density.

### Example 20

### Effect of Water Comprising Nanostructures on the Growth of Mesenchymal Stem Cells (MSCs)

MSCs are auto/paracrine cells (Caplan and Dennis 2006, J Cell Biochem 98(5): 1076-84), known to secrete factors that influence themselves and their surrounding cells. Gregory et al., (Gregory, Singh et al. 2003, J Biol Chem. 2003 Jul. 25; 278(30):28067-78. Epub 2003 May 9) have shown that cultured MSCs at 5 cells per cm² secrete dickkpof1 (DKK1) of the Wnt signaling pathway which enhance their proliferation. A similar effect can be achieved by adding 20% media from highly proliferating cells seeded at very low densities.

The following experiment was performed in order to determine the effect of Neowater™ on the growth of MSC's.

Materials and Methods

Cell culture: Human bone marrow (BM) cells were obtained from adult donors at the Laniado Hospital and Tel Aviv University, under approved protocols. They were cultured essentially as described. Briefly, 10-ml BM aspirates were taken from the iliac crust of male and female donors between the ages of 19-70. Mononuclear cells were isolated using a density gradient (ficoll/paque, Sigma) and resuspended in AMEM medium containing 25 mM glucose (all culture medium components were from Biological Industries, Beth Haemek, Israel, unless otherwise indicated) and supplemented with 16% FBS (lot no. CPB0183, Hyclone, Logan, Utah), 100 units/ml penicillin, 100 mg/ml streptomycin, and 2 mM L-glutamine. Cells were plated in 10-cm culture dishes (Corning, N.Y.), and incubated at 37° C. with 5% humidified CO₂. After 24 hours, nonadherent cells were discarded, and adherent cells were thoroughly washed twice with PBS. The cells were incubated for 5 to 7 days, harvested by treatment with 0.25% trypsin and 1 mM EDTA for 5 min at 37° C., seeded at 50-100 cells per cm² and cultured to confluence, termed passage 1. Cells from passage 1 were seeded in 24 well plates in densities of 50-100 cells per cm² and cultured in media based on Neowater™ or RO water, which was prepared out of powdered media (Biological industries, Beit Haemek, Israel 01-055-1A). The cell viability was assayed via crystal violet assay, once every 5 days for a total of 20 days. In addition, cells from one of the donor's were seeded in the above densities in 6 well plates (triplicates) and the cells were counted using a hemocytometer.

Results

3 bone marrow donors, one female and two male from passages 2-4 were grown at densities of 50-100 cells per cm² and assayed using cell count (FIG. 45) and crystal violet (FIG. 46).

Conclusion

Based on the data presented herein, the growth rate of stem cells (MSC's) in Neowater based media is enhanced at low cell density. When the cells reach high confluence the rate reduces and within 20 days, the amounts of cells in both conditions align. Gregory et al (Gregory, Singh et al. 2003, J Biol Chem. 2003 Jul. 25; 278(30):28067-78. Epub 2003 May 9), suggested that the rate of growth in MSC's are influenced through the autocrine secretion of DKK1. The lag period seen at the first 4-5 days in growth rates of the MSC's is due to the low concentration of DKK1. When reaching a high concentration of DKK1 in the growth media, the cells proliferate at high rates of up to 24-48 hours per doubling. The above data shows a shift in the growth period, implying that there is a higher concentration of DKK1 in the media in earlier periods. This phenomenon could be explained by the local concentration of DKK1 in the cell proximity, leading to enhanced proliferation.

### Example 21

### Cephalosporin Solubilization

The aim of the following experiments was to dissolve insoluble Cephalosporin in Neowater (NW) at a concentration of 3.6 mg/ml, using a slow solvent exchange procedure and to assess its bioactivity on E. Coli DH5α strain transformed with the Ampicillin (Amp) resistant bearing pUC19 plasmid.

Materials and Methods

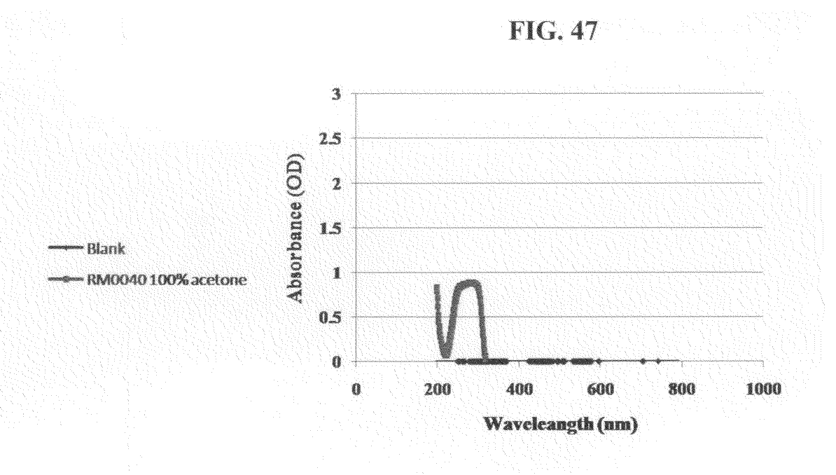

Slow solvent exchange: 25 mg of cephalosporin was dissolved in 5 ml organic solvent Acetone (5 mg/ml). Prior to addition of NW, the material was analyzed with a Heλios α spectrophotometer (FIG. 47. The material barely dissolved in acetone. It initially sedimented with a sand-like appearance. The procedure of exchanging the organic solvents with Neowater™ was performed on a multi block heater (set at 30° C.), and inside a desiccator and a hood. Organic solvent concentration was calculated according to the equations set forth in Table 7.

TABLE 7

Analytical Balance
% Acetone ml 1-0.1739X = Weighed value
% EtOH ml 1-0.2155X = Weighed value
Refractometer
% Acetone ml 0.0006X + 1.3328 = Refractive Index (RI) value
% EtOH ml 0.0006X + 1.3327 = Refractive Index (RI) value

Refractometer: RI: 1.3339, according to the equation calculations: 1.833%

Analytical balance: average: 0.9962, according to the equation: 1.941%.

The solution was filtered successfully using a 0.45 μm filter. Spectrophotometer readouts of the solution were performed before and after the filtration procedure.

Analysis of bioactivity of Cephalosporin dissolved in Neowater™: DH5α E.Coli bearing the pUC19 plasmid (Ampicillin resistant) were grown in liquid LB medium supplemented with 100 μg/ml ampicillin overnight at 37° C. and 220 rpm (Rounds per minute).

100 μL of the overnight (ON) starter re-inoculated in fresh liquid LB as follows:

a. 3 tubes with 100 μl of Neowater™: (only 2 experiment) and no antibiotics (both experiments).

b. 3 tubes with 10 μl of the Cephalosporin stock solution (50 ug/ml).

c. 3 tubes with 100 μl of the Cephalosporin stock solution (5 ug/ml).

Bacteria were incubated at 37° C. and 220 rpm. Sequential OD readings took place every hour using a 96 wells transparent plate with a 590 nm filter using the TECAN SPECTRAFlour Plus.

Results

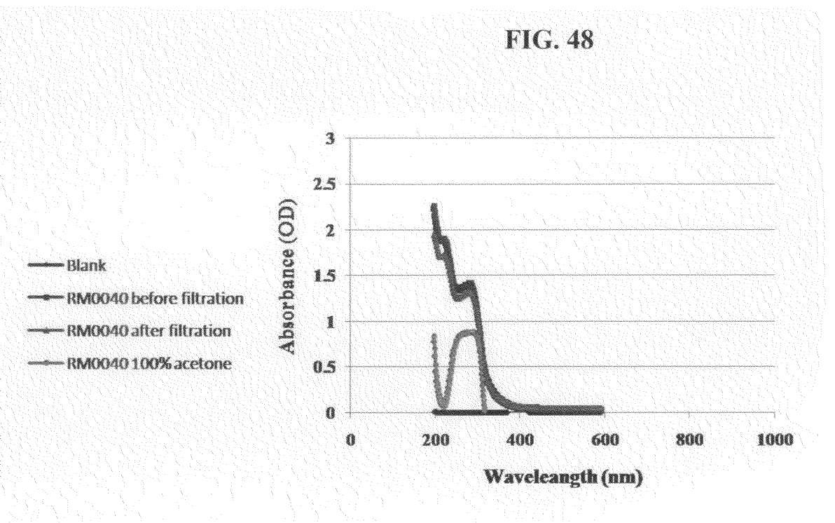

FIG. 48 is a spectrophotometer readout of Cephalosporin dissolved in Neowater™ prior to and following filtration.

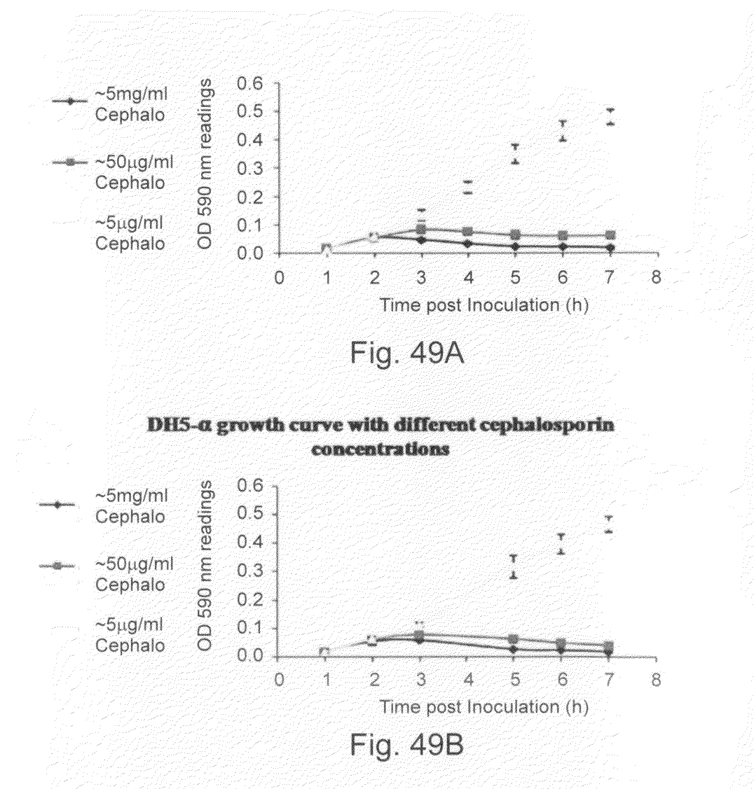

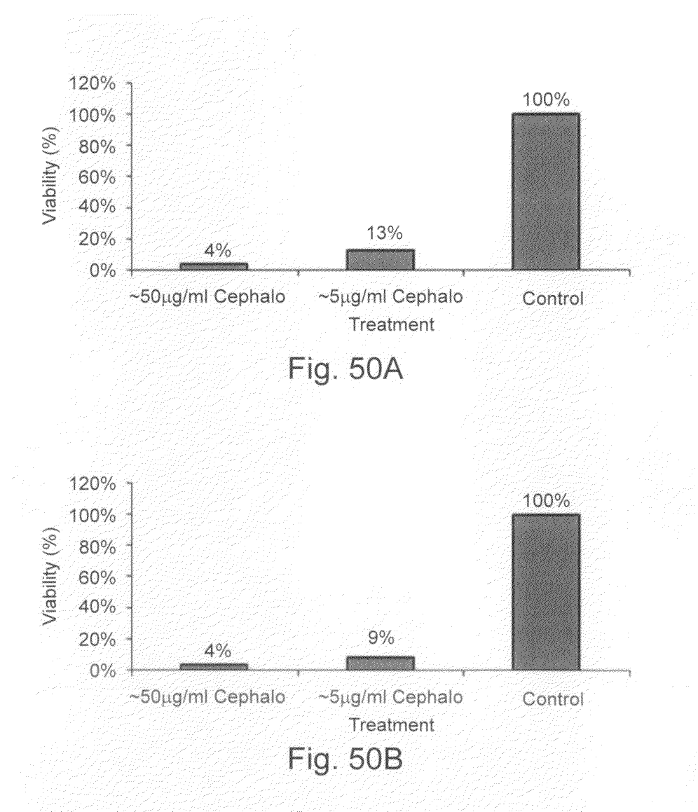

As illustrated in FIGS. 49A-B and 50A-B, when dissolved in Neowater™, Cephalosporin is bioavailable and bioactive as a bacterial growth inhibitor even when massively diluted. Of note, the present example teaches that Neowater™ itself has no role in bacterial growth inhibition.

### Example 22

### Optical Activity of Neowater™

Polarimetry measurements on the Neowater™ were devised to test signatures of induced long range order. The optical activity (in terms of circularly and elliptically polarized light) of the NPD solutions was measured using the Circular Dichroism (CD) method.

The Circular Dichroism (CD) experimental procedure: CD spectroscopy aims to detect absorption differences between left-handed and righthanded (L and R) polarized lights passed through aqueous solutions. Such differences can be generated from optically active (chiral) molecules immersed in water, distribution of molecules or nanoparticles or any other induced ordered structures in the water or solutions. The measurements reported here were performed using a Jasco K851 CD polarimeter at room temperature (298K). The spectrum was scanned between 190 nm and 280 nm using 1 nm and 10 seconds increments. In order to increase sensitivity and resolution a very long optical pathway was ensured by using 10 cm quartz cuvette (compared to 1 mm or smaller in regular mode of operation).

Results

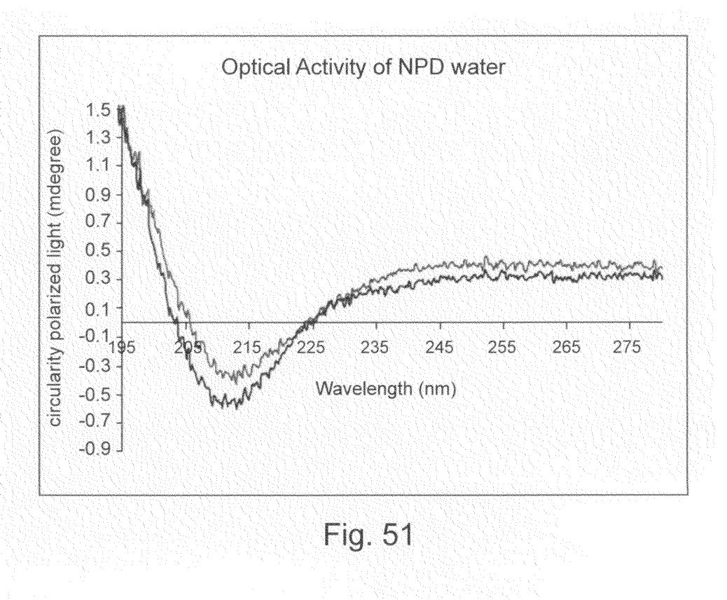

The results indicate that the Neowater™ shows circular dichroism. Two typical CD spectra performed in different batches of Neowater™, relative to the CD spectra of DDW (that was used as the baseline), are shown in FIG. 51. It is noted that the detected magnitude of the optical activity of about 0.5 millidegree is similar to the effect of 10⁵-10⁶ mole of ordinary peptide solution. Hence it is not a negligible level. CD measured differences in the absorption of left-handed polarized light versus right-handed polarized light arise due to structural asymmetry—The absence of regular structure results in vanishing CD intensity, while an ordered structure results in a spectrum which can contain positive and/or negative signals. Therefore, the present inventors propose that the existence of non vanishing signal in the CD spectra of the NPD solutions might be associated with the formation of long range orientational order in the Neowater™, formed by the network of nanoparticles and nanobubbles.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of cell-fusion, the method comprising fusing cells in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state, thereby fusing cells.

2-3. (canceled)

4. The method of claim 1, wherein said cells comprise primary cells.

5. The method of claim 1, wherein said cells comprise immortalized cells.

6. The method of claim 1, wherein said cells comprise tumor cells and antibody producing cells.

7. The method of claim 1, wherein said cells comprise stem cells and somatic cells.

8-12. (canceled)

13. The method of claim 6, wherein said tumor cells are incubated in said liquid composition for a period of time which allows an increase in hybridoma generation prior to said fusing.

14-22. (canceled)

23. A method of culturing eukaryotic cells, the method comprising incubating the cells in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state, thereby culturing eukaryotic cells.

24. (canceled)

25. The method of claim 23, wherein the eukaryotic cells are single cells.

26. The method of claim 25, wherein said single cell is a hybridoma.

27. The method of claim 23, wherein the culturing is effected in the absence of HCF.

28. The method of claim 23, wherein the eukaryotic cells are mesenchymal stem cells.

29-36. (canceled)

37. A cell culture medium comprising a eukaryotic cell culture medium and a liquid composition having a liquid and nanostructures, each of said nanostructures comprising a core material of a nanometric size surrounded by an envelope of ordered fluid molecules, said core material and said envelope of ordered fluid molecules being in a steady physical state.

38. The cell culture medium of claim 37, wherein said eukaryotic cell culture medium further comprises at least one agent selected from the group consisting of a growth factor, a serum and an antibiotic.

39-47. (canceled)

48. An article of manufacture comprising packaging material and a liquid composition identified for the culturing of eukaryotic cells being contained within said packaging material, said liquid composition having a liquid and nanostructures, each of said nanostructures comprising a core material of a nanometric size surrounded by an envelope of ordered fluid molecules, said core material and said envelope of ordered fluid molecules being in a steady physical state.

49. The article of manufacture of claim 48, wherein said eukaryotic cells are mesenchymal stem cells.

50-58. (canceled)

59. An article of manufacture comprising packaging material and a liquid composition identified for generating monoclonal antibodies being contained within said packaging material, said liquid composition having a liquid and nanostructures, each of said nanostructures comprising a core material of a nanometric size surrounded by an envelope of ordered fluid molecules, said core material and said envelope of ordered fluid molecules being in a steady physical state.

60-68. (canceled)

69. A method of generating a monoclonal antibody, the method comprising fusing an immortalizing cell with an antibody producing cell to obtain a hybridoma in a medium comprising a liquid composition having a liquid and nanostructures, each of said nanostructures comprising a core material of a nanometric size surrounded by an envelope of ordered fluid molecules, said core material and said envelope of ordered fluid molecules being in a steady physical state.

70. The method of claim 69, further comprising cloning said hybridoma.

71. The method of claim 70, wherein said cloning is effected by incubating said hybridoma in a medium comprising said liquid composition.

72. The method of claim 70, wherein said cloning is effected in the absence of HCF.

73. The method of claim 70, further comprising harvesting the monoclonal antibody following said cloning.

74-81. (canceled)

82. A method of dissolving or dispersing cephalosporin comprising contacting the cephalosporin with nanostructures and liquid under conditions which allow dispersion or dissolving of the substance, wherein said nanostructures comprise a core material of a nanometric size enveloped by ordered fluid molecules of said liquid, said core material and said envelope of ordered fluid molecules being in a steady physical state.
